SEQUENCE LISTING (38 sequences)

SEQ ID NO: 1 - protein, 10 aa, synthetic construct
  REGION 1..10: note = Synthetic Sequence
FWTEITDVNR

SEQ ID NO: 2 - protein, 10 aa, synthetic construct
  REGION 1..10: note = Synthetic Sequence
YETTTPSWNL

SEQ ID NO: 3 - protein, 18 aa, synthetic construct
  REGION 1..18: note = Synthetic Sequence
KVAVMTMAEANRRGTYSS

SEQ ID NO: 4 - protein, 132 aa, synthetic construct
  REGION 1..132: note = Synthetic Sequence
QVQLVESGGGVVQPGRSLRLSCAASFWTEITDVNRWFRQAPGKEREFVAYETTTPSWNLYADSVKGRFTISRDNSKNTLYLQMNSLRAEDTAVYYCARKVAVMTMAEANRRGTYSSWGQGTLVTVSSGPGGQ

SEQ ID NO: 5 - protein, 10 aa, synthetic construct
  REGION 1..10: note = Synthetic Sequence
FWTEITDVNR

SEQ ID NO: 6 - protein, 10 aa, synthetic construct
  REGION 1..10: note = Synthetic Sequence
WPGLFYAYDS

SEQ ID NO: 7 - protein, 18 aa, synthetic construct
  REGION 1..18: note = Synthetic Sequence
QTLAFAYNDSDWFEVYKG

SEQ ID NO: 8 - protein, 132 aa, synthetic construct
  REGION 1..132: note = Synthetic Sequence
QVQLVESGGGVVQPGRSLRLSCAASWMPVHDFSNFWFRQAPGKEREFVAEKFNMNPSDNYADSVKGRFTISRDNSKNTLYLQMNSLRAEDTAVYYCARKFNNHSMVPQWFHAIPLKWGQGTLVTVSSGPGGQ

SEQ ID NO: 9 - protein, 10 aa, synthetic construct
  REGION 1..10: note = Synthetic Sequence
WMPVHDFSNF

SEQ ID NO: 10 - protein, 10 aa, synthetic construct
  REGION 1..10: note = Synthetic Sequence
EKFNMNPSDN

SEQ ID NO: 11 - protein, 18 aa, synthetic construct
  REGION 1..18: note = Synthetic Sequence
KFNNHSMVPQWFHAIPLK

SEQ ID NO: 12 - protein, 132 aa, synthetic construct
  REGION 1..132: note = Synthetic Sequence
QVQLVESGGGVVQPGRSLRLSCAASFWTEITDVNRWFRQAPGKEREFVAWPGLFYAYDSYADSVKGRFTISRDNSKNTLYLQMNSLRAEDTAVYYCARQTLAFAYNDSDWFEVYKGWGQGTLVTVSSGPGGQ

SEQ ID NO: 13 - protein, 10 aa, synthetic construct
  REGION 1..10: note = Synthetic Sequence
FWTEITDVNR

SEQ ID NO: 14 - protein, 10 aa, synthetic construct
  REGION 1..10: note = Synthetic Sequence
YETTTPSWNL

SEQ ID NO: 15 - protein, 18 aa, synthetic construct
  REGION 1..18: note = Synthetic Sequence
SAELPDHHGDLFYVMKEN

SEQ ID NO: 16 - protein, 132 aa, synthetic construct
  REGION 1..132: note = Synthetic Sequence
QVQLVESGGGVVQPGRSLRLSCAASFWTEITDVNRWFRQAPGKEREFVAYETTTPSWNLYADSVKGRFTISRDNSKNTLYLQMNSLRAEDTAVYYCARSAELPDHHGDLFYVMKENWGQGTLVTVSSGPGGQ

SEQ ID NO: 17 - protein, 20 aa, synthetic construct
  REGION 1..20: note = Synthetic Sequence
QCYANHMYCNDSIAVYHFQM

SEQ ID NO: 18-20 - skipped (3 entries)

SEQ ID NO: 21 - protein, 10 aa, synthetic construct
  REGION 1..10: note = Synthetic Sequence
GGSGGSGGSG

SEQ ID NO: 22 - protein, 20 aa, synthetic construct
  REGION 1..20: note = Synthetic Sequence
LSSRPIQCHGLPCVLTSGLG

SEQ ID NO: 23 - protein, 20 aa, synthetic construct
  REGION 1..20: note = Synthetic Sequence
IRLILRNQVYCVSWQLSVIN

SEQ ID NO: 24 - protein, 20 aa, synthetic construct
  REGION 1..20: note = Synthetic Sequence
FLPRFFQWLCEPHWSADIVD

SEQ ID NO: 25 - protein, 20 aa, synthetic construct
  REGION 1..20: note = Synthetic Sequence
VYLDPCLVSLWTRSQVSIDG

SEQ ID NO: 26 - protein, 20 aa, synthetic construct
  REGION 1..20: note = Synthetic Sequence
WGVNQNVSGCTKLVDQRLLF

SEQ ID NO: 27 - protein, 20 aa, synthetic construct
  REGION 1..20: note = Synthetic Sequence
YIQGFRHMMVSDIPVVESFQ

SEQ ID NO: 28 - protein, 20 aa, synthetic construct
  REGION 1..20: note = Synthetic Sequence
SARYSLQVLRQLHCFSIDLI

SEQ ID NO: 29 - protein, 21 aa, synthetic construct
  REGION 1..21: note = Synthetic Sequence
RFCLGRYQFLINPQLHLTVYV

SEQ ID NO: 30 - protein, 20 aa, synthetic construct
  REGION 1..20: note = Synthetic Sequence
MHPQIHPDQTQFGNQGIRIA

SEQ ID NO: 31 - DNA, 396 bp, synthetic construct
  misc_feature 1..396: note = Synthetic Sequence
  source 1..396: mol_type = other DNA
caggtgcagctggtggagtctgggggaggcgtggtacagcctgggaggtccctgcgactctcctgtgcagcctcttggatgccggttcatgacttctctaacttctggttccgccaggctccagggaaggagcgtgagttcgtggcagaaaaattcaacatgaacccgtctgacaactacgcagactccgtgaagggccggttcaccatctcccgtgacaattccaagaacacgctgtatctgcaaatgaacagcctgcgtgccgaggacacggccgtgtattactgcgcgcgttctgctgaactgccggaccatcatggtgacctgttctacgttatgaaagaaaactggggtcaaggtaccctggtcaccgtctcctcaggaccgggaggacaa

SEQ ID NO: 32 - DNA, 396 bp, synthetic construct
  misc_feature 1..396: note = Synthetic Sequence
  source 1..396: mol_type = other DNA
caggtgcagctggtggagtctgggggaggcgtggtacagcctgggaggtccctgcgactctcctgtgcagcctctttctggactgaaatcactgacgttaaccgttggttccgccaggctccagggaaggagcgtgagttcgtggcatacgaaactactactccgtcttggaacctgtacgcagactccgtgaagggccggttcaccatctcccgtgacaattccaagaacacgctgtatctgcaaatgaacagcctgcgtgccgaggacacggccgtgtattactgcgcgcgtaaagttgctgttatgactatggctgaagctaaccgtcgtggtacttactcttcttggggtcaaggtaccctggtcaccgtctcctcaggaccgggaggacaa

SEQ ID NO: 33 - DNA, 396 bp, synthetic construct
  misc_feature 1..396: note = Synthetic Sequence
  source 1..396: mol_type = other DNA
caggtgcagctggtggagtctgggggaggcgtggtacagcctgggaggtccctgcgactctcctgtgcagcctcttggatgccggttcatgacttctctaacttctggttccgccaggctccagggaaggagcgtgagttcgtggcagaaaaattcaacatgaacccgtctgacaactacgcagactccgtgaagggccggttcaccatctcccgtgacaattccaagaacacgctgtatctgcaaatgaacagcctgcgtgccgaggacacggccgtgtattactgcgcgcgtaaattcaacaaccattctatggttccgcagtggttccatgctatcccgctgaaatggggtcaaggtaccctggtcaccgtctcctcaggaccgggaggacaa

SEQ ID NO: 34 - DNA, 396 bp, synthetic construct
  misc_feature 1..396: note = Synthetic Sequence
  source 1..396: mol_type = other DNA
caggtgcagctggtggagtctgggggaggcgtggtacagcctgggaggtccctgcgactctcctgtgcagcctctttctggactgaaatcactgacgttaaccgttggttccgccaggctccagggaaggagcgtgagttcgtggcatggccgggtctgttctacgcttacgactcttacgcagactccgtgaagggccggttcaccatctcccgtgacaattccaagaacacgctgtatctgcaaatgaacagcctgcgtgccgaggacacggccgtgtattactgcgcgcgtcagactctggctttcgcttacaacgactctgactggttcgaagtttacaaaggttggggtcaaggtaccctggtcaccgtctcctcaggaccgggaggacaa

SEQ ID NO: 35 - protein, 18 aa, synthetic construct
  REGION 1..18: note = Synthetic Sequence
SAELPDHHGDLFYVMKEN

SEQ ID NO: 36 - protein, 18 aa, synthetic construct
  REGION 1..18: note = Synthetic Sequence
KVAVMTMAEANRRGTYSS

SEQ ID NO: 37 - protein, 18 aa, synthetic construct
  REGION 1..18: note = Synthetic Sequence
KFNNHSMVPQWFHAIPLK

SEQ ID NO: 38 - protein, 18 aa, synthetic construct
  REGION 1..18: note = Synthetic Sequence
QTLAFAYNDSDWFEVYKG

(19) United States
(12) Patent Application Publication
(10) Pub. No.: US 2024/0368278 A1
(43) Pub. Date: Nov. 7, 2024

(54) GENERATION AND CHARACTERIZATION OF NOVEL TIM-4 BINDING AGENTS

(71) Applicant: Heat Biologics, Inc., Morrisville, NC (US)

(72) Inventors: Karla FRIETZE, Morrisville, NC (US); Padmini JAYARAMAN, Morrisville, NC (US); Matthew SEAVEY, Morrisville, NC (US); John PRENDERGAST, Morrisville, NC (US); Neil GOLDSTEIN, Morrisville, NC (US); Jeffrey WOLF, Morrisville, NC (US)

(21) Appl. No.: 18/682,284

(22) PCT Filed: Aug. 9, 2022
(86) PCT No.: PCT/US2022/074712

§ 371 Date: Feb. 8, 2024

Related U.S. Application Data
(60) Provisional application No. 63/231,455, filed on Aug. 10, 2021.

(51) Int. Cl. C07K 16/28

(52) U.S. Cl. CPC C07K 16/2803; C07K 2317/565; C07K 2317/567; C07K 2317/622

(57) ABSTRACT

The disclosure is directed to compositions that specifically bind to the Tim-4, and related methods of use.

### CROSS-REFERENCE TO RELATED APPLICATIONS

This application claims priority to and benefit of U.S. Provisional Patent Application No. 63/231,455 filed on Aug. 10, 2021, the contents of all of which are hereby incorporated by reference in its entirety.

### FIELD OF THE DISCLOSURE

The disclosure is directed to compositions that specifically bind to T-cell immunoglobulin and mucin domain containing 4 (Tim-4), and related methods.

### DESCRIPTION OF THE TEXT FILE SUBMITTED ELECTRONICALLY

This application contains a Sequence Listing in XML format submitted electronically herewith via EFS-Web. Said XML copy, created on Aug. 9, 2021, is named SKX-002 Sequence Listing and is 47,466 bytes in size. The Sequence Listing is incorporated herein by reference in its entirety.

### BACKGROUND

T-cell immunoglobulin and mucin domain containing 4 (Tim-4) is, inter alia, a phosphatidylserine receptor which is selectively expressed on, e.g., antigen presenting cells. Tim-4 has been demonstrated to be expressed on iNKT cells, B1 cells, and tumor cells. To date, studies have pointed to various biological functions of the molecule, including participation in immune-related diseases. For instance, Tim-4 may play a substantial role in maintaining homeostasis by regulating various immune responses, including viral infection, allergy, autoimmunity, and tumor immunity.

Thus, there remains a need for agents that modulate the activity of Tim-4.

### SUMMARY

Accordingly, in various aspects, the present disclosure relates to compositions which specifically bind to Tim-4. Further, in various aspects, the present disclosure relates to methods of using compositions which specifically bind to Tim-4, e.g. for the treatment or prevention of cancers or autoimmune diseases.

In various aspects, the present disclosure relates to a composition which specifically binds to Tim-4, wherein the composition is or comprises: (a) a single-chain antibody, or fragment thereof comprising CDR1, CDR2, and CDR3, wherein: CDR1 is FWTEITDVNR (SEQ ID NO: 1) or FWTEITDVNR (SEQ ID NO: 5), or WMPVHDFSNF (SEQ ID NO: 9), or FWTEITDVNR (SEQ ID NO: 13), or a variant thereof; CDR2 is YETTTPSWNL (SEQ ID NO: 2) or WPGLFYAYDS (SEQ ID NO: 6), or EKFNMNPSDN (SEQ ID NO: 10), or YETTTPSWNL (SEQ ID NO: 14), or a variant thereof; and CDR3 is KVAVMTMAEANRRGTYSS (SEQ ID NO: 3) or QTLAFAYNDSDWFEVYKG (SEQ ID NO: 7), or KFNNHSMVPQWFHAIPLK (SEQ ID NO: 11), or SAELPDHHGDLFYVMKEN (SEQ ID NO: 15), or a variant thereof; or (b) a peptide, and the peptide is selected from: QCYANHMYCNDSIAVYHFQM (SEQ ID NO: 17), or a variant thereof; LSSRPIQCHGLPCVLTSGLG (SEQ ID NO: 22), or a variant thereof; MHPQIHPDQTQFGNQGIRIA (SEQ ID NO: 30), or a variant thereof, IRLILRNQVYCVSWQLSVIN (SEQ ID NO: 23), or a variant thereof, FLPRFFQWLCEPHWSADIVD (SEQ ID NO: 24), or a variant thereof, VYLDPCLVSLWTRSQVSIDG (SEQ ID NO: 25), or a variant thereof, WGVNQNVSGCTKLVDQRLLF (SEQ ID NO: 26), or a variant thereof, YIQGFRHMMVSDIPVVESFQ (SEQ ID NO: 27), or a variant thereof, SARYSLQVLRQLHCFSIDLI (SEQ ID NO: 28), or a variant thereof, RFCLGRYQFLINPQLHLTVYV (SEQ ID NO: 29), or a variant thereof, SAELPDHHGDLFYVMKEN (SEQ ID NO: 35), or a variant thereof; KVAVMTMAEANRRGTYSS (SEQ ID NO: 36), or a variant thereof; KFNNHSMVPQWFHAIPLK (SEQ ID NO: 37), or a variant thereof; QTLAFAYNDSDWFEVYKG (SEQ ID NO: 38), or a variant thereof.

In some embodiments, CDR1 is FWTEITDVNR (SEQ ID NO: 1), or a variant thereof, CDR2 is YETTTPSWNL (SEQ ID NO: 2), or a variant thereof, and CDR3 is KVAVMTMAEANRRGTYSS (SEQ ID NO: 3), or a variant thereof, wherein the variant has about 1, or about 2, or about 3, or about 4, or about 5 mutations, the mutations selected from substitutions or deletions.

In some embodiments, CDR1 is FWTEITDVNR (SEQ ID NO: 5), or a variant thereof, CDR2 is WPGLFYAYDS (SEQ ID NO: 6), or a variant thereof, and CDR3 is QTLAFAYNDSDWFEVYKG (SEQ ID NO: 7), or a variant thereof, wherein the variant comprises about 1, or about 2, or about 3, or about 4, or about 5 mutations, the mutations selected from substitutions or deletions.

In some embodiments, CDR1 is WMPVHDFSNF (SEQ ID NO: 9), or a variant thereof, CDR2 is EKFNMNPSDN (SEQ ID NO: 10), or a variant thereof, and CDR3 is KFNNHSMVPQWFHAIPLK (SEQ ID NO: 11), or a variant thereof, wherein the variant has about 1, or about 2, or about 3, or about 4, or about 5 mutations, the mutations selected from substitutions or deletions.

In some embodiments, CDR1 is FWTEITDVNR (SEQ ID NO: 13), or a variant thereof, CDR2 is YETTTPSWNL (SEQ ID NO: 14), or a variant thereof, and CDR3 is SAELPDHHGDLFYVMKEN (SEQ ID NO: 15), or a variant thereof, wherein the variant comprises about 1, or about 2, or about 3, or about 4, or about 5 mutations, the mutations selected from substitutions or deletions.

In some embodiments, the single-chain antibody, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

In some embodiments, the variable region FW sequences are human.

In some embodiments, the single-chain antibody comprises an amino acid sequence of: QVQLVESGGGVVQPGRSLRLSCAASFWTEITDVNRWFRQAPGKEREFVAYETTTPS WNLYADSVKGRFTISRDNSKNTLYLQMNSLRAEDTAVYYCARKVAVMTMAEANR RGTYSSWGQGTLVTVSSGPGGQ (SEQ ID NO: 4), or an amino acid sequence having at least about 90%, or at least about 95%, or at least about 97%, or at least about 98%, or at least about 99% identity with SEQ ID NO: 4.

In some embodiments, the single-chain antibody comprises an amino acid sequence of: QVQLVESGGGVVQPGRSLRLSCAASWMPVHDFSNFWFRQAPGKEREFVAEKFNMN PSDNYADSVKGRFTISRDNSKNTLYLQMNSLRAEDTAVYYCARKFNNHSMVPQWF HAIPLKWGQGTLVTVSSGPGGQ (SEQ ID NO: 8), or an amino acid sequence having at least about 90%, or at least about 95%, or at least about 97%, or at least about 98%, or at least about 99% identity with SEQ ID NO: 8.

In some embodiments, the single-chain antibody comprises an amino acid sequence of: QVQLVESGGGVVQPGRSLRLSCAASFWTEITDVNRWFRQAPGKEREFVAWPGLFYA YDSYADSVKGRFTISRDNSKNTLYLQMNSLRAEDTAVYYCARQTLAFAYNDSDWFE VYKGWGQGTLVTVSSGPGGQ (SEQ ID NO: 12), or an amino acid sequence having at least about 90%, or at least about 95%, or at least about 97%, or at least about 98%, or at least about 99% identity with SEQ ID NO: 12.

In some embodiments, the single-chain antibody comprises an amino acid sequence of: QVQLVESGGGVVQPGRSLRLSCAASFWTEITDVNRWFRQAPGKEREFVAYETTTPS WNLYADSVKGRFTISRDNSKNTLYLQMNSLRAEDTAVYYCARSAELPDHHGDLFY VMKENWGQGTLVTVSSGPGGQ (SEQ ID NO: 16), or an amino acid sequence having at least about 90%, or at least about 95%, or at least about 97%, or at least about 98%, or at least about 99% identity with SEQ ID NO: 16.

In some embodiments, the peptide is or comprises: QCYANHMYCNDSIAVYHFQM (SEQ ID NO: 17), or a variant thereof, wherein the variant comprises about 1, or about 2, or about 3, or about 4, or about 5 mutations, the mutations selected from substitutions or deletions.

In some embodiments, the peptide is or comprises: SAELPDHHGDLFYVMKEN (SEQ ID NO: 35), or a variant thereof; wherein the variant comprises about 1, or about 2, or about 3, or about 4, or about 5 mutations, the mutations selected from substitutions or deletions.

In some embodiments, the peptide is or comprises: KVAVMTMAEANRRGTYSS (SEQ ID NO: 36), or a variant thereof; wherein the variant comprises about 1, or about 2, or about 3, or about 4, or about 5 mutations, the mutations selected from substitutions or deletions.

In some embodiments, the peptide is or comprises: KFNNHSMVPQWFHAIPLK (SEQ ID NO: 37), or a variant thereof; wherein the variant comprises about 1, or about 2, or about 3, or about 4, or about 5 mutations, the mutations selected from substitutions or deletions.

In some embodiments, the peptide is or comprises: QTLAFAYNDSDWFEVYKG (SEQ ID NO: 38), or a variant thereof; wherein the variant comprises about 1, or about 2, or about 3, or about 4, or about 5 mutations, the mutations selected from substitutions or deletions.

In some embodiments, the peptide is or comprises: LSSRPIQCHGLPCVLTSGLG (SEQ ID NO: 22), or a variant thereof; wherein the variant comprises about 1, or about 2, or about 3, or about 4, or about 5 mutations, the mutations selected from substitutions or deletions.

In some embodiments, the peptide is or comprises: MHPQIHPDQTQFGNQGIRIA (SEQ ID NO: 30), or a variant thereof; wherein the variant comprises about 1, or about 2, or about 3, or about 4, or about 5 mutations, the mutations selected from substitutions or deletions.

In some embodiments, the peptide is or comprises: IRLILRNQVYCVSWQLSVIN (SEQ ID NO: 23), or a variant thereof; wherein the variant comprises about 1, or about 2, or about 3, or about 4, or about 5 mutations, the mutations selected from substitutions or deletions.

In some embodiments, the peptide is or comprises: FLPRFFQWLCEPHWSADIVD (SEQ ID NO: 24), or a variant thereof; wherein the variant comprises about 1, or about 2, or about 3, or about 4, or about 5 mutations, the mutations selected from substitutions or deletions.

In some embodiments, the peptide is or comprises: VYLDPCLVSLWTRSQVSIDG (SEQ ID NO: 25), or a variant thereof; wherein the variant comprises about 1, or about 2, or about 3, or about 4, or about 5 mutations, the mutations selected from substitutions or deletions.

In some embodiments, the peptide is or comprises: WGVNQNVSGCTKLVDQRLLF (SEQ ID NO: 26), or a variant thereof; wherein the variant comprises about 1, or about 2, or about 3, or about 4, or about 5 mutations, the mutations selected from substitutions or deletions.

In some embodiments, the peptide is or comprises: YIQGFRHMMVSDIPVVESFQ (SEQ ID NO: 27), or a variant thereof; wherein the variant comprises about 1, or about 2, or about 3, or about 4, or about 5 mutations, the mutations selected from substitutions or deletions.

In some embodiments, the peptide is or comprises: SARYSLQVLRQLHCFSIDLI (SEQ ID NO: 28), or a variant thereof; wherein the variant comprises about 1, or about 2, or about 3, or about 4, or about 5 mutations, the mutations selected from substitutions or deletions.

In some embodiments, the peptide is or comprises: RFCLGRYQFLINPQLHLTVYV (SEQ ID NO: 29), or a variant thereof; wherein the variant comprises about 1, or about 2, or about 3, or about 4, or about 5 mutations, the mutations selected from substitutions or deletions.

In various embodiments, the peptide further comprises an additional peptide.

In various embodiments, the composition comprises a dimer of peptides, and in some embodiments, the composition comprises a trimer of peptides. In some embodiments, peptides are joined with a linker which is substantially comprised of glycine and serine residues. In some embodiments, the linker is or comprises (GGS)ₙ, wherein ₙ is 1, or 2, or 3, or 4, or 5. For example, in some embodiments, the linker is or comprises GGSGGSGGSG (SEQ ID NO: 21), or a variant thereof, wherein the variant comprises about 1, or about 2, or about 3, or about 4, or about 5 mutations, the mutations selected from substitutions or deletions.

In various embodiments, the composition further comprises a targeting moiety. For example, in some embodiments, the targeting moiety is directed to a tumor cell. In some embodiments, the targeting moiety is directed to a tumor-associated antigen (TAA), such as HER2, PSA, TRP-2, EpCAM, GPC3, mesothelin (MSLN), and EGFR.

In various embodiments, the present disclosure provides a polynucleotide comprising a nucleic acid sequence encoding the single-chain antibody, or a fragment thereof or a peptide.

In some embodiments, the present disclosure provides a vector, or a host cell, comprising the polynucleotide having a nucleic acid sequence encoding the single-chain antibody, or a fragment thereof or a peptide.

In aspects, the present disclosure provides a pharmaceutical composition comprising the composition of any of the preceding embodiments, and a pharmaceutically acceptable excipient or carrier.

In aspects, the present disclosure provides a method for treating or preventing cancer, comprising administering an effective amount of composition of any one of the present embodiments to a patient in need thereof. In some embodiments, the cancer is a solid tumor or a blood cancer. In some embodiments, the cancer is selected form one or more a cancer of a blood vessel, an eye tumor, of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In aspects, the present disclosure provides a method for treating or preventing an autoimmune disease or disorder, comprising administering an effective amount of the composition of any one of the present embodiments to a patient in need thereof. In some embodiments, the autoimmune disease or disorder is selected from graft versus host disease, transplantation rejection (e.g., prevention of allograft rejection), multiple sclerosis, diabetes mellitus, lupus, celiac disease, Crohn's disease, ulcerative colitis, Guillain-Barre syndrome, scleroderma, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Meniere's syndrome; pernicious anemia, rheumatoid arthritis, systemic lupus erythematosus, dermatomyositis, Sjogren's syndrome, lupus erythematosus, multiple sclerosis, myasthenia gravis, Reiter's syndrome, and Grave's disease.

In some embodiments, the autoimmune disease or disorder is graft versus host disease.

In aspects, the present disclosure provides a method for inducing phagocytosis, e.g. of apoptotic or tumor cells, comprising administering an effective amount of composition of any one of the present embodiments to a patient in need thereof.

In aspects, the present disclosure provides a method for inducing or increasing phagocytosis, e.g. of apoptotic and/or tumor cells, comprising administering an effective amount of composition of any one of the present embodiments to a patient in need thereof.

In aspects, the present disclosure provides a method for inhibiting or reducing phagocytosis, e.g. of apoptotic and/or tumor cells, comprising administering an effective amount of composition of any one of the present embodiments to a patient in need thereof.

In aspects, the present disclosure provides a method for inducing or increasing autophagy, e.g. of apoptotic and/or tumor cells, comprising administering an effective amount of composition of any one of the present embodiments to a patient in need thereof.

In aspects, the present disclosure provides a method for inhibiting or reducing autophagy, e.g. of apoptotic and/or tumor cells, comprising administering an effective amount of composition of any one of the present embodiments to a patient in need thereof.

In aspects, the present disclosure provides a method for inducing or increasing T cell proliferation, e.g. of apoptotic or tumor cells, comprising administering an effective amount of composition of any one of the present embodiments to a patient in need thereof or contacting cells with a composition of any one of the present embodiments (e.g. ex vivo, e.g. with allogeneic or autologous cells).

In aspects, the present disclosure provides a method for inhibiting or reducing binding of Tim-4 with one or more binding partners (e.g., without limitation AMPKα1 or Tim-1, or phosphatidylserine (PS), or leukocyte mono-immunoglobulin (Ig)-like receptor 5 (LMIR5)), comprising administering an effective amount of composition of any one of the present embodiments to a patient in need thereof or contacting cells with a composition of any one of the present embodiments (e.g. ex vivo, e.g. with allogeneic or autologous cells).

In aspects, the present disclosure provides a method for inhibiting or reducing tumor growth and/or inducing or increasing immune tolerance comprising administering an effective amount of composition of any one of the present embodiments to a patient in need thereof or contacting cells with a composition of any one of the present embodiments (e.g. ex vivo, e.g. with allogeneic or autologous cells).

In aspects, the present disclosure provides a method for inhibiting or reducing Tim-4 binding of T cells, e.g. activated T cells, comprising administering an effective amount of composition of any one of the present embodiments to a patient in need thereof or contacting cells with a composition of any one of the present embodiments (e.g. ex vivo, e.g. with allogeneic or autologous cells).

In aspects, the present disclosure provides a method for increasing immune tolerance comprising administering an effective amount of composition of any one of the present embodiments to a patient in need thereof or contacting cells with a composition of any one of the present embodiments. In embodiments, the increase in immune tolerance increases transplant tolerance.

In aspects, the present disclosure provides a method of decreasing, inhibiting, suppressing, ameliorating, or delaying a Th1-mediated immune response comprising administering an effective amount of composition of any one of the present embodiments to a patient in need thereof or contacting cells with a composition of any one of the present embodiments. In embodiments, the increase in immune tolerance increases transplant tolerance.

The details of one or more examples of the disclosure are set forth in the description below. Other features or advantages of the present disclosure will be apparent from the following drawings, detailed description of several examples, and also from the appended claims. The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### BRIEF DESCRIPTION OF THE DRAWINGS

FIG. 1 is an image of a CLUSTAL alignment showing the CDR3 regions of antibodies that bound to Tim-4.

FIG. 2A, FIG. 2B, and FIG. 2C show an image of the THP-1 experimental workflow, an image of THP-1 cells undergoing phagocytosis, and a graph showing data from the phagocytosis experiment. FIG. 2A is an image of the experimental workflow. FIG. 2B is a graph showing data from the peptides derived from the CDR3 regions of antibodies tested in the phagocytosis assay. In FIG. 2B, along the x-axis, “156-1G” refers to SEQ ID NO: 7, “156-IH” refers to SEQ ID NO: 11, “156-2B” refers to SEQ ID NO: 3, and “156-2F” refers to SEQ ID NO: 15. In this assay, cells were treated with the indicated treatments, and cytochalasin D (20 μM) was used as a control to show phagocytosis failure. CDR3 regions from antibodies that bound to Tim-4 were synthesized as peptides, and subsequently tested. The data shown in FIG. 2B represents peptides tested at 100 μg/mL. Statistics were performed using a one-way ANOVA followed by a multiple comparison test comparing each treatment to “untreated” control (p>0.05=*, ns=not significant). FIG. 2C shows representative microscopic images of THP-1 cells undergoing phagocytosis. Phosphatidylserine (“PS”) lipid microparticles are shown and superimposed onto brightfield images of THP-1 cells. 156-2B and 156-1G significantly inhibited phagocytosis in THP-1 cells. Scale bar represents 150 μm.

FIG. 3A and FIG. 3B are graphs showing dose response curves of the phagocytosis assay in THP-1 cells using both PS beads and carboxylate beads. FIG. 3A shows a dose response curve of the peptide SEQ ID NO: 17 derived from the CDR3 of an antibody that bound Tim-4 using phosphatidyl serine microparticles (PS beads). FIG. 3B shows a dose response curve of the peptide SEQ ID NO: 17 derived from the CDR3 of an antibody that bound Tim-4 using fluorescent Carboxylate-Modified Microspheres, 1.0 μm, crimson fluorescent (625/645) beads.

FIG. 4A and FIG. 4B shows images demonstrating Tim4-Hek293-FT cell line validation. FIG. 4A shows HEK293-FT cells infected with lentiviral Tim-4 particles. Stable Tim-4 expression was confirmed by RT-PCR using primers against Tim-4 mRNA and Gapdh mRNA (control). FIG. 4B shows a representative image of Tim4-Hek293-FT cells ability to phagocytose using carboxylate beads. Scale bar represents 300 μm.

FIG. 5A and FIG. 5B shows a graph and image of a peptide derived from the CDR3 of SEQ ID NO: 3, and the capability of SEQ ID NO: 3 to limit phagocytosis in the Tim4-Hek293-FT cell line. In FIG. 5A, the Tim4-Hek293-FT cells were plated in a 96 well plate, followed by the phagocytosis assay. Statistical analysis was performed using a t-test (p>0.05=*). Relative fluorescence units (“RFU”) were normalized to fluorescent intensity at hour zero. In FIG. 5B, Tim4-Hek293-FT cells were fixed and immunolabeled against Tim-4 and counterstained with DAPI. FIG. 5B is a representative image showing a reduced ability of phagocytosis of the phosphatidylserine bead in cells treated with SEQ ID NO: 17. Arrows indicate phosphatidylserine bead. Scale bar represents 75 μm.

FIG. 6 is an image of a CLUSTAL alignment showing the identification of 20mer peptides that bound to Tim-4.

FIG. 7A and FIG. 7B shows a graph and image of a Tim4-Hek293-FT phagocytosis assay using 20mer peptides that bound to Tim4. In FIG. 7A, cells were plated on a 96 well plate and treatments were performed similar to FIG. 2A. Statistics were performed using a one-way ANOVA followed by a multiple comparison test comparing each treatment to the “untreated” control (p>0.05=*, p>0.01=**, p>0.001=***). Additional statistically significant changes were demonstrated between treatments, but for ease of viewing only statistical differences between “untreated” and SEQ ID NO: 17 is shown in FIG. 7A. In FIG. 7A, “116-117 1A” is SEQ ID NO: 30 (MHPQIHPDQTQFGNQGIRIA), “116-117 2C” is SEQ ID NO: 23 (IRLILRNQVYCVSWQLSVIN), “116-117 2D” is SEQ ID NO: 24 (FLPRFFQWLCEPHWSADIVD), “116-117 2F” is SEQ ID NO: 25 (VYLDPCLVSLWTRSQVSIDG), “116-117 3B” is SEQ ID NO: 26 (WGVNQNVSGCTKLVDQRLLF), “116-117 3C” is SEQ ID NO: 27 (YIQGFRHMMVSDIPVVESFQ), “116-117 3E” is SEQ ID NO: 17 (QCYANHMYCNDSIAVYHFQM), “116-117 3F” is SEQ ID NO: 28 (SARYSLQVLRQLHCFSIDLI), “116-117 4A” is SEQ ID NO: 22 (LSSRPIQCHGLPCVLTSGLG), and “116-117 4G” is SEQ ID NO: 29 (RFCLGRYQFLINPQLHLTVYV). FIG. 7B shows immunofluorescence images of cells fixed with anti-TIM4 and DAPI counterstain. 116-117 3E inhibits phagocytosis in Tim4-Hek293-FT cells. Scale bar represents 150 μm.

FIG. 8A, FIG. 8B, FIG. 8C, and FIG. 8D shows graphs and images of a THP-1 cell phagocytosis assay using a 20mer peptide (SEQ ID NO: 17) that bound to Tim4. FIG. 8A shows a graph of a THP-1 cell phagocytosis assay. In FIG. 8A, cells were plated on a 96 well plate and treatments were performed with carboxylate beads. Statistics were performed using a one-way ANOVA followed by a multiple comparison test comparing each treatment to the “untreated” control (p>0.05=*, p>0.01=**, p>0.001=***, p>0.0001=****). FIG. 8B are representative images of the phagocytosis assay. 116-117 3E inhibits phagocytosis in THP-1 cells. Scale bare represents 150 μm. FIG. 8C shows immunofluorescence experiments performed on fixed cells with anti-TIM4 and DAPI counterstain. 116-117 3E inhibits phagocytosis in THP-1 cells. Scale bar represents 150 μm. FIG. 8D is a graph of a dose response curve.

FIG. 9A and FIG. 9B shows graphs of a RAW phagocytosis assay (FIG. 9A) using RAW 264.7 cells, and a dose response curve of a RAW phagocytosis assay (FIG. 9B). In FIG. 9A, cells were plated on a 96 well plate and treatments were performed with carboxylate beads. Statistics were performed using a one-way ANOVA, followed by a multiple comparison test comparing each treatment to the “untreated” control (p>0.05=*, p>0.01=**, p>0.001=***, p>0.0001=****). In FIG. 9B, a dose response curve of a RAW phagocytosis assay was performed using mouse macrophages RAW cells with cytochalasin D as a control, and graphed in log scale.

FIG. 10A, FIG. 10B, and FIG. 10C shows graphs of a Hek293FT-Tim4 phagocytosis assay (FIG. 10A), a THP-1 phagocytosis assay (FIG. 10B), and a RAW phagocytosis assay using mouse macrophages RAW 264.7 cells (FIG. 10C). In FIG. 10A, FIG. 10B, and FIG. 10C, cells were plated on a 96 well plate and treatments were performed with carboxylate beads. Statistics were performed using a one-way ANOVA followed by a multiple comparison test comparing each treatment to “untreated” control (p>0.05=*, p>0.01=**, p>0.001=***, p>0.0001=****).

FIG. 11A and FIG. 11B shows images of how the 20mer peptide (SEQ ID NO: 17) reduces autophagosome formation in THP-1 cells. FIG. 11A shows the experimental workflow, and FIG. 11B shows representative images. In FIG. 11B, autophagosomes are labeled and Hoechst stain is used for nuclear staining.

### DETAILED DESCRIPTION

The present invention is based, in part, on the surprising discovery of a composition that specifically binds T-cell immunoglobulin and mucin domain containing 4 (Tim-4). The composition disclosed herein can be a small protein, or a peptide, or an antibody (e.g., a single-chain antibody), that binds to Tim-4. The compositions disclosed herein are capable of functioning as agonists or antagonists. In addition, the present disclosure describes peptide, small protein, and antibody compositions that are interchangeable. The peptide or small protein can be derived from the binding region of an antibody (e.g., a single-chain antibody), and target specific peptides and small proteins can be incorporated into the binding regions of the antibody (e.g., a single-chain antibody). The compositions disclosed herein may be used in a method of treating or preventing cancer (e.g., a solid tumor, or blood cancer), or an autoimmune disease (e.g., graft versus host disease).

In various embodiments, disclosed herein is a composition that specifically binds to Tim-4.

In various embodiments, disclosed herein is a composition which specifically binds to Tim-4 on the surface of a cell. In various embodiments, disclosed herein is a composition which specifically binds to Tim-4 on the surface of myeloid cells. In various embodiments, disclosed herein is a composition which specifically binds to Tim-4 on the surface of antigen presenting cells. In various embodiments, disclosed herein is a composition which specifically binds to Tim-4 on the surface of T cells. In various embodiments, disclosed herein is a composition which specifically binds to Tim-4 on the surface of dendritic cells. In various embodiments, disclosed herein is a composition which specifically binds to Tim-4 on the surface of macrophages.

In various embodiments, disclosed herein is a composition which specifically binds to human Tim-4.

In various embodiments, disclosed herein is a composition which specifically binds to murine Tim-4.

In various embodiments, disclosed herein is a composition which specifically binds to Tim-4, preferentially over e.g. Tim-1 or Tim-3. In embodiments, the composition specifically binds to Tim-4 with an affinity of greater than about 10-fold, or greater than about 50-fold, or greater than about 100-fold, or greater than about 300-fold, or greater than about 500-fold, or greater than about 1000-fold, or greater than about 3000-fold greater than binding to Tim-1 or Tim-3.

In various embodiments, disclosed herein is a composition which specifically binds to a particular antigen, e.g., Tim-4, when it binds to that antigen in a sample, and does not recognize and bind, or recognizes and binds to a lesser extent, other molecules in the sample. In some embodiments, the compositions of the present disclosure can selectively bind to an epitope with an affinity (K_{d}) equal to or less than, for example, about 1×10⁻⁶ M (e.g., equal to or less than about 1×10⁻⁹ M, equal to or less than about 1×10⁻¹⁰ M, equal to or less than about 1×10⁻¹¹ M, or equal to or less than about 1×10⁻¹² M) in phosphate buffered saline. The ability of a compositions of the present disclosure to specifically bind a protein epitope can be determined using any of the methods known in the art or those methods described herein (e.g., by Biacore/Surface Plasmon Resonance). This can include, for example, binding to Tim-4 on live cells as a method to stimulate caspase activation in live transformed cells, binding to an immobilized target substrate including human Tim-4 fusion proteins as detected using an ELISA method, binding to Tim-4 on live cells as detected by flow cytometry, or binding to an immobilized substrate by surface plasmon resonance (including ProteOn).

In some embodiments, the composition disclosed herein is a peptide, a small protein, or an antibody (e.g., a single-chain antibody), that binds to Tim-4.

In embodiments, a single-chain antibody is a single-chain variable fragment (scFv).

In embodiments, the composition disclosed herein is a single-chain antibody, small protein, or a single polypeptide that contains at least one variable binding domain (e.g., a variable domain of a mammalian heavy or light chain immunoglobulin, a camelid VHH, or a cartilaginous fish (e.g., shark) Ig-NAR domain) that is capable of specifically binding to an antigen. Non-limiting examples of single-chain antibodies include single-domain antibodies.

As used herein, the term “single-domain antibody” refers to a polypeptide that contains one camelid VHH or at least one cartilaginous fish Ig-NAR domain that is capable of specifically binding to an antigen. Non-limiting examples of single-domain antibodies are described, for example, in U.S. Publication No. 2010/0092470.

In some embodiments, the small protein ranges in size from about 2 kDa to about 4 kDa. In some embodiments, the small protein ranges in size from about 2 kDa to about 3.5 kDa, from about 2 kDa to about 3 kDa, or from about 2 kDa to about 2.5 kDa.

In some embodiments, the peptide or small protein is an agonist. In some embodiments, the peptide or small protein is an antagonist.

In some embodiments, the antibody ranges in size from about 5 kDa to about 30 kDa, from about 5 kDa to about 25 kDa, from about 5 kDa to about 20 kDa, or from about 5 kDa to about 15 kDa. In some embodiments, the antibody (e.g., a single-chain antibody) is a 12 kDa fragment.

In some embodiments, the antibody retains the biological function of the parent, whole antibody. In some embodiments, the small protein and antibody compositions are interchangeable. In some embodiments, the peptide or small protein is derived from the binding region of the antibody. In some embodiments, the target specific small protein is incorporated into the binding regions of the antibody (e.g., a single-chain antibody).

In embodiments, a single-chain antibody is a single-chain variable fragment (scFv). In embodiments, the composition disclosed herein is a single-chain antibody, small protein, or a single polypeptide that contains at least one variable binding domain (e.g., a variable domain of a mammalian heavy or light chain immunoglobulin, a camelid VHH, or a cartilaginous fish (e.g., shark) Ig-NAR domain) that is capable of specifically binding to an antigen. Non-limiting examples of single-chain antibodies include single-domain antibodies.

In various embodiments, the composition comprises a single-chain antibody, or fragment thereof, having a heavy chain variable domain. The variable domain determines the specificity of the antibody. Each variable region comprises three hypervariable regions also known as complementarity determining regions (CDRs) flanked by four relatively conserved framework regions (FRs). The three CDRs, referred to as CDR1, CDR2, and CDR3, contribute to the antibody binding specificity. In some embodiments, the single-chain antibody is a chimeric antibody. In some embodiments, the single-chain antibody is a humanized antibody. In some embodiments, the single-chain antibody, or fragment thereof comprises a CDR1, CDR2, and CDR3, wherein:

| | CDR1 is |
|---|---|
| (SEQ ID NO: 1) | |
| | FWTEITDVNR |
| | or |
| (SEQ ID NO: 5) | |
| | FWTEITDVNR, |
| | or |
| (SEQ ID NO: 9) | |
| | WMPVHDFSNF, |
| | or |
| (SEQ ID NO: 13) | |
| | FWTEITDVNR, or a variant thereof; |
| | CDR2 is |
| (SEQ ID NO: 2) | |
| | YETTTPSWNL |
| | or |
| (SEQ ID NO: 6) | |
| | WPGLFYAYDS, |
| | or |
| (SEQ ID NO: 10) | |
| | EKFNMNPSDN, |
| | or |
| (SEQ ID NO: 14) | |
| | YETTTPSWNL, or a variant thereof; |
| | and |
| | CDR3 is |
| (SEQ ID NO: 3) | |
| | KVAVMTMAEANRRGTYSS |
| | or |
| (SEQ ID NO: 7) | |
| | QTLAFAYNDSDWFEVYKG, |
| | or |
| (SEQ ID NO: 11) | |
| | KFNNHSMVPQWFHAIPLK, |
| | or |
| (SEQ ID NO: 15) | |
| | SAELPDHHGDLFYVMKEN, or a variant |
| | thereof. |

In some embodiments, the composition comprises a peptide, and the peptide is selected from:

(SEQ ID NO: 17)

QCYANHMYCNDSIAVYHFQM, or a variant thereof;

(SEQ ID NO: 22)

LSSRPIQCHGLPCVLTSGLG, or a variant thereof;

(SEQ ID NO: 30)

MHPQIHPDQTQFGNQGIRIA, or a variant thereof,

(SEQ ID NO: 23)

IRLILRNQVYCVSWQLSVIN, or a variant thereof,

(SEQ ID NO: 24)

FLPRFFQWLCEPHWSADIVD, or a variant thereof,

(SEQ ID NO: 25)

VYLDPCLVSLWTRSQVSIDG, or a variant thereof,

(SEQ ID NO: 26)

WGVNQNVSGCTKLVDQRLLF, or a variant thereof,

(SEQ ID NO: 27)

YIQGFRHMMVSDIPVVESFQ, or a variant thereof,

(SEQ ID NO: 28)

SARYSLQVLRQLHCFSIDLI, or a variant thereof,

(SEQ ID NO: 29)

RFCLGRYQFLINPQLHLTVYV, or a variant thereof,

(SEQ ID NO: 35)

SAELPDHHGDLFYVMKEN, or a variant thereof;

(SEQ ID NO: 36)

KVAVMTMAEANRRGTYSS, or a variant thereof;

(SEQ ID NO: 37)

KFNNHSMVPQWFHAIPLK, or a variant thereof;
and

(SEQ ID NO: 38)

QTLAFAYNDSDWFEVYKG, or a variant thereof.

In some embodiments, CDR1 is FWTEITDVNR (SEQ ID NO: 1), or a variant thereof, CDR2 is YETTTPSWNL (SEQ ID NO: 2), or a variant thereof, and CDR3 is KVAVMTMAEANRRGTYSS (SEQ ID NO: 3), or a variant thereof, wherein the variant has about 1, or about 2, or about 3, or about 4, or about 5 mutations, the mutations selected from substitutions or deletions.

In some embodiments, CDR1 is FWTEITDVNR (SEQ ID NO: 5), or a variant thereof, CDR2 is WPGLFYAYDS (SEQ ID NO: 6), or a variant thereof, and CDR3 is QTLAFAYNDSDWFEVYKG (SEQ ID NO: 7), or a variant thereof, wherein the variant comprises about 1, or about 2, or about 3, or about 4, or about 5 mutations, the mutations selected from substitutions or deletions.

In some embodiments, CDR1 is WMPVHDFSNF (SEQ ID NO: 9), or a variant thereof, CDR2 is EKFNMNPSDN (SEQ ID NO: 10), or a variant thereof, and CDR3 is KFNNHSMVPQWFHAIPLK (SEQ ID NO: 11), or a variant thereof, wherein the variant has about 1, or about 2, or about 3, or about 4, or about 5 mutations, the mutations selected from substitutions or deletions.

In some embodiments, CDR1 is FWTEITDVNR (SEQ ID NO: 13), or a variant thereof, CDR2 is YETTTPSWNL (SEQ ID NO: 14), or a variant thereof, and CDR3 is SAELPDHHGDLFYVMKEN (SEQ ID NO: 15), or a variant thereof, wherein the variant comprises about 1, or about 2, or about 3, or about 4, or about 5 mutations, the mutations selected from substitutions or deletions.

In some embodiments, the single-chain antibody, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

In some embodiments, the variable region FW sequences are human.

In some embodiments, the single-chain antibody comprises an amino acid sequence of: QVQLVESGGGVVQPGRSLRLSCAASFWTEITDVNRWFRQAPGKEREFVAYETTTPS WNLYADSVKGRFTISRDNSKNTLYLQMNSLRAEDTAVYYCARKVAVMTMAEANR RGTYSSWGQGTLVTVSSGPGGQ (SEQ ID NO: 4), or an amino acid sequence having at least about 90%, or at least about 95%, or at least about 97%, or at least about 98%, or at least about 99% identity with SEQ ID NO: 4.

In some embodiments, the single-chain antibody comprises an amino acid sequence of: QVQLVESGGGVVQPGRSLRLSCAASWMPVHDFSNFWFRQAPGKEREFVAEKFNMN PSDNYADSVKGRFTISRDNSKNTLYLQMNSLRAEDTAVYYCARKFNNHSMVPQWF HAIPLKWGQGTLVTVSSGPGGQ (SEQ ID NO: 8), or an amino acid sequence having at least about 90%, or at least about 95%, or at least about 97%, or at least about 98%, or at least about 99% identity with SEQ ID NO: 8.

In some embodiments, the single-chain antibody comprises an amino acid sequence of: QVQLVESGGGVVQPGRSLRLSCAASFWTEITDVNRWFRQAPGKEREFVAWPGLFYA YDSYADSVKGRFTISRDNSKNTLYLQMNSLRAEDTAVYYCARQTLAFAYNDSDWFE VYKGWGQGTLVTVSSGPGGQ (SEQ ID NO: 12), or an amino acid sequence having at least about 90%, or at least about 95%, or at least about 97%, or at least about 98%, or at least about 99% identity with SEQ ID NO: 12.

In some embodiments, the single-chain antibody comprises an amino acid sequence of: QVQLVESGGGVVQPGRSLRLSCAASFWTEITDVNRWFRQAPGKEREFVAYETTTPS WNLYADSVKGRFTISRDNSKNTLYLQMNSLRAEDTAVYYCARSAELPDHHGDLFY VMKENWGQGTLVTVSSGPGGQ (SEQ ID NO: 16), or an amino acid sequence having at least about 90%, or at least about 95%, or at least about 97%, or at least about 98%, or at least about 99% identity with SEQ ID NO: 16.

In some embodiments, the peptide is or comprises: QCYANHMYCNDSIAVYHFQM (SEQ ID NO: 17), or a variant thereof, wherein the variant comprises about 1, or about 2, or about 3, or about 4, or about 5 mutations, the mutations selected from substitutions or deletions.

In some embodiments, the peptide is or comprises: LSSRPIQCHGLPCVLTSGLG (SEQ ID NO: 22), or a variant thereof; wherein the variant comprises about 1, or about 2, or about 3, or about 4, or about 5 mutations, the mutations selected from substitutions or deletions.

In some embodiments, the peptide is or comprises: MHPQIHPDQTQFGNQGIRIA (SEQ ID NO: 30), or a variant thereof; wherein the variant comprises about 1, or about 2, or about 3, or about 4, or about 5 mutations, the mutations selected from substitutions or deletions.

In some embodiments, the peptide is or comprises: IRLILRNQVYCVSWQLSVIN (SEQ ID NO: 23), or a variant thereof; wherein the variant comprises about 1, or about 2, or about 3, or about 4, or about 5 mutations, the mutations selected from substitutions or deletions.

In some embodiments, the peptide is or comprises: FLPRFFQWLCEPHWSADIVD (SEQ ID NO: 24), or a variant thereof; wherein the variant comprises about 1, or about 2, or about 3, or about 4, or about 5 mutations, the mutations selected from substitutions or deletions.

In some embodiments, the peptide is or comprises: VYLDPCLVSLWTRSQVSIDG (SEQ ID NO: 25), or a variant thereof; wherein the variant comprises about 1, or about 2, or about 3, or about 4, or about 5 mutations, the mutations selected from substitutions or deletions.

In some embodiments, the peptide is or comprises: WGVNQNVSGCTKLVDQRLLF (SEQ ID NO: 26), or a variant thereof; wherein the variant comprises about 1, or about 2, or about 3, or about 4, or about 5 mutations, the mutations selected from substitutions or deletions.

In some embodiments, the peptide is or comprises: YIQGFRHMMVSDIPVVESFQ (SEQ ID NO: 27), or a variant thereof; wherein the variant comprises about 1, or about 2, or about 3, or about 4, or about 5 mutations, the mutations selected from substitutions or deletions.

In some embodiments, the peptide is or comprises: SARYSLQVLRQLHCFSIDLI (SEQ ID NO: 28), or a variant thereof; wherein the variant comprises about 1, or about 2, or about 3, or about 4, or about 5 mutations, the mutations selected from substitutions or deletions.

In some embodiments, the peptide is or comprises: RFCLGRYQFLINPQLHLTVYV (SEQ ID NO: 29), or a variant thereof; wherein the variant comprises about 1, or about 2, or about 3, or about 4, or about 5 mutations, the mutations selected from substitutions or deletions.

In some embodiments, the peptide is or comprises: SAELPDHHGDLFYVMKEN (SEQ ID NO: 35), or a variant thereof; wherein the variant comprises about 1, or about 2, or about 3, or about 4, or about 5 mutations, the mutations selected from substitutions or deletions.

In some embodiments, the peptide is or comprises: KVAVMTMAEANRRGTYSS (SEQ ID NO: 36), or a variant thereof; wherein the variant comprises about 1, or about 2, or about 3, or about 4, or about 5 mutations, the mutations selected from substitutions or deletions.

In some embodiments, the peptide is or comprises: KFNNHSMVPQWFHAIPLK (SEQ ID NO: 37), or a variant thereof; wherein the variant comprises about 1, or about 2, or about 3, or about 4, or about 5 mutations, the mutations selected from substitutions or deletions.

In some embodiments, the peptide is or comprises: QTLAFAYNDSDWFEVYKG (SEQ ID NO: 38), or a variant thereof; wherein the variant comprises about 1, or about 2, or about 3, or about 4, or about 5 mutations, the mutations selected from substitutions or deletions.

In various embodiments, the peptide further comprises an additional peptide. In some embodiments, the composition comprises a dimer of peptides. In some embodiments, the composition comprises a trimer of peptides.

In some embodiments, the peptides are joined with a linker which is substantially comprised of glycine and serine residues. In some embodiments, the linker is (GGS)n, wherein n is 1, or 2, or 3, or 4, or 5. In some embodiments, the linker is GGSGGSGGSG (SEQ ID NO: 21), or a variant thereof, wherein the variant comprises about 1, or about 2, or about 3, or about 4, or about 5 mutations, the mutations selected from substitutions or deletions.

In various embodiments, the present compositions comprise a nucleic acid sequence having at least about 70%, at least about 75%, at least about 80%, at least about 85%, at least about 90%, at least about 95%, at least about 96%, at least about 97%, at least about 98%, or at least about 99% similarity to the nucleic acid sequences of any one of SEQ ID NOs: 31, 32, 33, or 34, or a codon-optimized version thereof.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the composition further comprises a targeting moiety. In some embodiments, the targeting moiety is directed to a tumor cell. In some embodiments, the targeting moiety is directed to a tumor-associated antigen (TAA). In some embodiments, the TAA is selected from HER2, PSA, TRP-2, EpCAM, GPC3, mesothelin (MSLN), and EGFR.

In some embodiments, the present disclosure provides a polynucleotide comprising a nucleic acid sequence encoding the single-chain antibody, or a fragment thereof, or peptide described in any of the preceding embodiments.

In some embodiments, the polynucleotide is RNA or DNA. In some embodiments, the RNA is a messenger RNA (mRNA) or a modified mRNA.

In some embodiments, a vector comprising the polynucleotide is provided. In some embodiments, a host cell comprising the vector is provided. Cells may be cultured in vitro or genetically engineered, for example. Host cells can be obtained from normal or affected subjects, including healthy humans, cancer patients, and patients with an infectious disease, private laboratory deposits, public culture collections such as the American Type Culture Collection, or from commercial suppliers.

In various embodiments, the present disclosure provides a pharmaceutical composition comprising the composition of any of the embodiments disclosed herein, and a pharmaceutically acceptable excipient.

### Methods of Treating or Preventing Diseases

In some embodiments, the compositions of the disclosure find use in the treatment or prevention of various diseases or disorders.

In some embodiments, the compositions of the disclosure inhibit the engulfment of apoptotic cells by macrophages following administration of the composition to a subject.

In some embodiments, the compositions of the disclosure stimulate or increase pro-inflammatory cytokines administration to a subject. In some embodiments, the pro-inflammatory cytokines comprise at least one of IL-1β, IL6, and TNFα

In some embodiments, the methods disclosed herein treat or prevent cancer by administering an effective amount of the composition that specifically binds to Tim-4. In various embodiments, the composition comprises a single-chain antibody, or fragment thereof, having a heavy chain variable domain. In some embodiments, the single-chain antibody, or fragment thereof comprises a CDR1, CDR2, and CDR3, wherein: CDR1 is FWTEITDVNR (SEQ ID NO: 1) or FWTEITDVNR (SEQ ID NO: 5), or WMPVHDFSNF (SEQ ID NO: 9), or FWTEITDVNR (SEQ ID NO: 13), or a variant thereof; CDR2 is YETTTPSWNL (SEQ ID NO: 2) or WPGLFYAYDS (SEQ ID NO: 6), or EKFNMNPSDN (SEQ ID NO: 10), or YETTTPSWNL (SEQ ID NO: 14), or a variant thereof; and CDR3 is KVAVMTMAEANRRGTYSS (SEQ ID NO: 3) or QTLAFAYNDSDWFEVYKG (SEQ ID NO: 7), or KFNNHSMVPQWFHAIPLK (SEQ ID NO: 11), or SAELPDHHGDLFYVMKEN (SEQ ID NO: 15), or a variant thereof.

In some embodiments, the composition comprises a peptide, and the peptide is selected from: QCYANHMYCNDSIAVYHFQM (SEQ ID NO: 17), or a variant thereof; LSSRPIQCHGLPCVLTSGLG (SEQ ID NO: 22), or a variant thereof; MHPQIHPDQTQFGNQGIRIA (SEQ ID NO: 30), or a variant thereof, IRLILRNQVYCVSWQLSVIN (SEQ ID NO: 23), or a variant thereof, FLPRFFQWLCEPHWSADIVD (SEQ ID NO: 24), or a variant thereof, VYLDPCLVSLWTRSQVSIDG (SEQ ID NO: 25), or a variant thereof. WGVNQNVSGCTKLVDQRLLF (SEQ ID NO: 26), or a variant thereof, YIQGFRHMMVSDIPVVESFQ (SEQ ID NO: 27), or a variant thereof. SARYSLQVLRQLHCFSIDLI (SEQ ID NO: 28), or a variant thereof, RFCLGRYQFLINPQLHLTVYV (SEQ ID NO: 29), or a variant thereof. SAELPDHHGDLFYVMKEN (SEQ ID NO: 35), or a variant thereof, KVAVMTMAEANRRGTYSS (SEQ ID NO: 36), or a variant thereof, KFNNHSMVPQWFHAIPLK (SEQ ID NO: 37), or a variant thereof, and QTLAFAYNDSDWFEVYKG (SEQ ID NO: 38), or a variant thereof.

In some embodiments, CDR1 is FWTEITDVNR (SEQ ID NO: 1), or a variant thereof, CDR2 is YETTTPSWNL (SEQ ID NO: 2), or a variant thereof, and CDR3 is KVAVMTMAEANRRGTYSS (SEQ ID NO: 3), or a variant thereof, wherein the variant has about 1, or about 2, or about 3, or about 4, or about 5 mutations, the mutations selected from substitutions or deletions.

In some embodiments, CDR1 is FWTEITDVNR (SEQ ID NO: 5), or a variant thereof, CDR2 is WPGLFYAYDS (SEQ ID NO: 6), or a variant thereof, and CDR3 is QTLAFAYNDSDWFEVYKG (SEQ ID NO: 7), or a variant thereof, wherein the variant comprises about 1, or about 2, or about 3, or about 4, or about 5 mutations, the mutations selected from substitutions or deletions.

In some embodiments, CDR1 is WMPVHDFSNF (SEQ ID NO: 9), or a variant thereof, CDR2 is EKFNMNPSDN (SEQ ID NO: 10), or a variant thereof, and CDR3 is KFNNHSMVPQWFHAIPLK (SEQ ID NO: 11), or a variant thereof, wherein the variant has about 1, or about 2, or about 3, or about 4, or about 5 mutations, the mutations selected from substitutions or deletions.

In some embodiments, CDR1 is FWTEITDVNR (SEQ ID NO: 13), or a variant thereof, CDR2 is YETTTPSWNL (SEQ ID NO: 14), or a variant thereof, and CDR3 is SAELPDHHGDLFYVMKEN (SEQ ID NO: 15), or a variant thereof, wherein the variant comprises about 1, or about 2, or about 3, or about 4, or about 5 mutations, the mutations selected from substitutions or deletions.

In some embodiments, the methods disclosed herein prevent an onset or progression of cancer by administering an effective amount a composition disclosed herein (e.g., a single-chain antibody or peptide) to the subject.

As a non-limiting example, the prevention of an onset, the presence, and/or the evaluation of the progression of a cancer in a subject can be assessed according to the Tumor/Nodes/Metastases (TNM) system of classification (International Union Against Cancer, 6th edition, 2002), or the Whitmore-Jewett staging system (American Urological Association). Typically, cancers are staged using a combination of physical examination, blood tests, and medical imaging. If tumor tissue is obtained via biopsy or surgery, examination of the tissue under a microscope can also provide pathologic staging. In some embodiments, the stage or grade of a cancer assists a practitioner in determining the prognosis for the cancer and in selecting the appropriate modulating therapy.

In some embodiments, the prevention of an onset, or progression, of cancer is assessed using the overall stage grouping as a non-limiting example: Stage I cancers are localized to one part of the body, typically in a small area; Stage II cancers are locally advanced and have grown into nearby tissues or lymph nodes, as are Stage III cancers. Whether a cancer is designated as Stage II or Stage III can depend on the specific type of cancer. The specific criteria for Stages II and III can differ according to diagnosis. Stage IV cancers have often metastasized or spread to other organs or throughout the body. The onset or progression of cancer can be assessed using conventional methods available to one of skill in the art, such as a physical exam, blood tests, and imaging scans (e.g., X-rays, MRI, CT scans, ultrasound etc.).

As disclosed herein, administering, or administering a treatment/therapy, refers to a treatment/therapy from which a subject receives a beneficial effect, such as the reduction, decrease, attenuation, diminishment, stabilization, remission, suppression, inhibition or arrest of the development or progression of cancer, or a symptom thereof.

In some embodiments, the treatment/therapy that a subject receives, or the prevention in the onset of cancer results in at least one or more of the following effects: (1) the reduction or amelioration of the severity of cancer and/or a genetic disease or disorder, and/or a symptom associated therewith; (2) the reduction in the duration of a symptom associated with cancer and/or a genetic disease or disorder; (3) the prevention in the recurrence of a symptom associated with cancer and/or a genetic disease or disorder; (4) the regression of cancer and/or a genetic disease or disorder, and/or a symptom associated therewith; (5) the reduction in hospitalization of a subject; (6) the reduction in hospitalization length; (7) the increase in the survival of a subject; (8) the inhibition of the progression of cancer and/or a genetic disease or disorder and/or a symptom associated therewith; (9) the enhancement or improvement the therapeutic effect of another therapy; (10) a reduction or elimination in the cancer cell population, and/or a cell population associated with a genetic disease or disorder; (11) a reduction in the growth of a tumor or neoplasm; (12) a decrease in tumor size; (13) a reduction in the formation of a tumor; (14) eradication, removal, or control of primary, regional and/or metastatic cancer; (15) a decrease in the number or size of metastases; (16) a reduction in mortality; (17) an increase in cancer-free survival rate of a subject; (18) an increase in relapse-free survival; (19) an increase in the number of subjects in remission; (20) a decrease in hospitalization rate; (21) the size of the tumor is maintained and does not increase in size or increases the size of the tumor by less than about 5% or about 10% after administration of a therapy as measured by conventional methods available to one of skill in the art, e.g., X-rays, MRI, CAT scan, ultrasound etc.; (22) the prevention of the development or onset of cancer and/or a genetic disease or disorder, and/or a symptom associated therewith; (23) an increase in the length of remission for a subject; (24) the reduction in the number of symptoms associated with cancer and/or a genetic disease or disorder; (25) an increase in symptom-free survival of a cancer subject and/or a subject associated with a genetic disease or disorder; and/or (26) limitation of or reduction in metastasis. In some embodiments, the treatment/therapy that a subject receives does not cure cancer, but prevents the progression or worsening of the disease. In certain embodiments, the treatment/therapy that a subject receives does not prevent the onset/development of cancer, but may prevent the onset of cancer symptoms.

In some embodiments, “preventing” an onset or progression of cancer in a subject in need thereof, is inhibiting or blocking the cancer or disorder. In some embodiments, the methods disclosed herein prevent, or inhibit, the cancer or disorder at any amount or level. In some embodiments, the methods disclosed herein prevent or inhibit the cancer or genetic disease or disorder by at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition, or at least or about a 100% inhibition).

In various embodiments, disclosed herein is a method for treating or preventing cancer, comprising administering an effective amount of the composition of any one of the above claims to a patient in need thereof. In some embodiments, the cancer is a solid tumor. In some embodiments, the cancer is a blood cancer. In some embodiments, the cancer is selected form one or more of a cancer of a blood vessel, an eye tumor, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In aspects, the present disclosure provides a method of increasing anti-tumor immunity in a subject having cancer comprising administering an effective amount of composition of any one of the present embodiments to a patient in need thereof or contacting cells with a composition of any one of the present embodiments (e.g. ex vivo, e.g. with allogeneic or autologous cells).

In embodiments, the anti-tumor immunity includes the processing and presentation of released antigens by antigen-presenting cells (APCs), interaction with T lymphocytes, subsequent immune/T-cell activation, trafficking of antigen-specific effector cells, and the engagement of the target tumor cell by the activated effector T cell. Anti-tumor immunity can be imposed by antigen-specific CD8+ T cells and tumoricidal macrophages. Antigens (Ag), typically foreign substances of environmental, viral, or bacterial origin, products of somatically altered proteins, or debris from dying (apoptotic) cells are processed and presented by major histocompatibility complex (MHC) on antigen presenting cells, including (but not limited to) dendritic cells, macrophages, and B cells. CD8+ T cells utilize T cell receptors (TCRs) to recognize WIC-presented peptides and subsequently mount an antigen-specific cytolytic attack. In particular, Ag-TCR engagement ultimately leads to activation and proliferation of CD8+ T cells that play a crucial role in autoimmunity, response to pathogens, and tumor suppression. Also, myeloid cells, including various subsets of monocytes, neutrophils, and macrophages, are implicated in T cell suppression.

In embodiments, increased anti-tumor immunity can be identified by measuring at least one of: increased CD8+ T cells, increased CD4+ T cells, increased CD4+ or CD8+ T cells secreting interferon gamma, and/or decreased CD4+ T cells secreting IL10 among tumor-infiltrating lymphocytes.

In embodiments, the subject has a cancer that interacts with Tim-4 on the surface of myeloid cells, thereby decreasing anti-tumor immunity compared to a subject without cancer or a subject with a cancer that does not interact with Tim-4. In particular embodiments, interaction with Tim-4 or Tim-4 activity can be detected in the microenvironment of the tumor.

Myeloid cells in the tumor microenvironment (TME), including M2 macrophages, N2 neutrophils and myeloid-derived suppressor cells (MDSC), play a critical role in immune suppression by directly causing functional exhaustion of the cytotoxic T cells or by indirectly increasing the suppressive power of T-regulatory cells (Tregs). This leads to a skewed immunostimulatory versus immunosuppressive balance in the TME. The immunostimulatory environment of the TME is largely shaped by the presence of cytotoxic T cells and NK cells, cytolytic and phagocytosis-inducing M1 macrophages, cytotoxic N1 neutrophils, humoral response inducing B cells, and antigen presenting immunogenic dendritic cells (DC). Immunostimulatory cytokines and chemokines such as interferon gamma (IFN-γ), interleukin-12 (IL-12), tumor necrosis factor-alpha (TNF-α), etc. are key coordinators of the immunostimulatory activity. Important molecules that bias the immunosuppressive nature of the TME are anti-inflammatory Th2 cells, N2 neutrophils, M2 macrophages, Tregs, and tolerogenic DC. Immunosuppressive cytokines and chemokines such as transforming growth factor-beta (TGF-β), interleukin-10) (IL-10), macrophage colony stimulating factor (M-CSF), interleukin-4 (IL-4), etc. are key coordinators of the immunosuppressive activity. In embodiments, the Th1 response in a tumor microenvironment is increased by administration of one or more of the present compositions.

As described herein, the “tumor microenvironment” (TME) is the surrounding microenvironment that constantly interacts with tumor cells which is conducive to allow cross-talk between tumor cells and its environment. A tumor microenvironment plays a role in disrupting the cancer immunity cycle and plays a critical role in multiple aspects of cancer progression. For example, the TME can decrease drug penetration, confer proliferative and anti-apoptotic advantages to surviving cells, facilitate resistance without causing genetic mutations and epigenetic changes, and collectively modify disease modality and distort clinical indices. Without being limiting, the tumor microenvironment can include the cellular environment of the tumor, surrounding blood vessels, immune cells, fibroblasts, bone marrow derived inflammatory cells, lymphocytes, signaling molecules and the extracellular matrix. The tumor environment can include tumor cells or malignant cells that are aided and influenced by the tumor microenvironment to ensure growth and survival. The tumor microenvironment can also include tumor-infiltrating immune cells such as lymphoid and myeloid cells, which can stimulate or inhibit the antitumor immune response and stromal cells such as tumor-associated fibroblasts and endothelial cells that contribute to the tumor's structural integrity. Without being limiting, stromal cells can include cells that make up tumor-associated blood vessels, such as endothelial cells and pericytes, which are cells that contribute to structural integrity (fibroblasts), as well as tumor-associated macrophages (TAMs) and infiltrating immune cells including monocytes, neutrophils (PMN), dendritic cells (DCs), T and B cells, mast cells, and natural killer (NK) cells. The stromal cells make up the bulk of tumor cellularity while the dominating cell type in solid tumors is the macrophage.

In embodiments, blocking of Tim-4 on the surface of myeloid cells can increase M1 macrophage polarization. For example, the M1 macrophage production can be increased by at least about about 10%, about 20%, about 30%, about 40%, about 50%, about 60%, about 70%, about 80%, about 90%, about 100%, about 150%, about 200% or greater when compared to the M1 macrophage level prior to administration of one or more of the present compositions.

Macrophages may be classified by subsets: classically (M1) or alternatively (M2) activated macrophages (see, e.g., Laskin, Chem Res Toxicol. 2009 Aug. 17; 22 (8): 1376-1385, the contents of which are hereby incorporated by reference in their entireties). Without wishing to be bound by theory, M1 macrophages are activated by standard mechanisms, such as IFNγ, LPS, and TNFα, while M2 macrophages are activated by alternative mechanisms, such as IL-4, IL-13, IL-10, and TGFβ. M1 macrophages can display a cytotoxic, proinflammatory phenotype, while M2 macrophages, suppress some aspects of immune and inflammatory responses and participate in wound repair and angiogenesis. In embodiments, administration of one or more of the present compositions increases the level of M1 macrophages. In embodiments, administration of an one or more of the present compositions increases the level of M1 macrophages in the tumor microenvironment.

In specific embodiments, the uptake of apoptotic cells is reduced following administration of one or more of the present compositions increases. In specific embodiment, uptake of apoptotic cells is reduced by at least about 5%, about 10%, about 20%, about 30%, about 40%, about 50%, about 60%, about 70%, about 80%, about 90%, about 95%, about 96%, about 97%, about 98%, about 99%, about 100%, about 5-10%, about 10-20%, about 10-30%, about 10-40%, about 20-30%, about 20-40%, about 30-40%, about 30-50%, about 40-50%, about 40-60%, about 50-60%, about 50-70%, about 60-70%, about 60-80%, about 70-80%, about 70-90%, about 80-90%, about 80-100%, about 90-100%, or about 95-100%.

In embodiments, blocking of Tim-4 can reduce pulmonary metastasis or micrometastases. For example, pulmonary metastasis or micrometastases can be reduced by about 5%, about 10%, about 20%, about 30%, about 40%, about 50%, about 60%, about 70%, about 80%, about 90%, about 100%, about 150%, about 200% or greater when compared to the pulmonary metastasis or micrometastases level prior to administration of one or more of the present compositions.

In various embodiments, disclosed herein is a method for treating or preventing an autoimmune disease or disorder, comprising administering an effective amount of the composition of any one of the above claims to a patient in need thereof. In some embodiments, the autoimmune disease or disorder is selected from graft versus host disease, transplantation rejection (e.g., prevention of allograft rejection), multiple sclerosis, diabetes mellitus, lupus, celiac disease, Crohn's disease, ulcerative colitis, Guillain-Barre syndrome, scleroderma, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Meniere's syndrome; pernicious anemia, rheumatoid arthritis, systemic lupus erythematosus, dermatomyositis, Sjogren's syndrome, lupus erythematosus, multiple sclerosis, myasthenia gravis, Reiter's syndrome, and Grave's disease. In some embodiments, the autoimmune disease or disorder is graft versus host disease.

In aspects, the present disclosure provides a method for inducing phagocytosis, e.g. of apoptotic or tumor cells, comprising administering an effective amount of composition of any one of the present embodiments to a patient in need thereof.

In aspects, the present disclosure provides a method for inducing or increasing phagocytosis, e.g. of apoptotic or tumor cells, comprising administering an effective amount of composition of any one of the present embodiments to a patient in need thereof.

In aspects, the present disclosure provides a method for inhibiting or reducing phagocytosis, e.g. of apoptotic or tumor cells, comprising administering an effective amount of composition of any one of the present embodiments to a patient in need thereof.

In aspects, the present disclosure provides a method for inducing or increasing T cell proliferation, e.g. of apoptotic or tumor cells, comprising administering an effective amount of composition of any one of the present embodiments to a patient in need thereof or contacting cells with a composition of any one of the present embodiments (e.g. ex vivo, e.g. with allogeneic or autologous cells).

In aspects, the present disclosure provides a method for inhibiting or reducing binding of Tim-4 with one or more binding partners (e.g., without limitation AMPKα1 or Tim-1, or phosphatidylserine (PS), or leukocyte mono-immunoglobulin (Ig)-like receptor 5 (LMIR5)), comprising administering an effective amount of composition of any one of the present embodiments to a patient in need thereof or contacting cells with a composition of any one of the present embodiments (e.g. ex vivo, e.g. with allogeneic or autologous cells).

In aspects, the present disclosure provides a method for inhibiting or reducing tumor growth and/or inducing or increasing immune tolerance comprising administering an effective amount of composition of any one of the present embodiments to a patient in need thereof or contacting cells with a composition of any one of the present embodiments (e.g. ex vivo, e.g. with allogeneic or autologous cells).

In aspects, the present disclosure provides a method of reducing the uptake of cells by myeloid cells, e.g. apoptotic cells, e.g. macrophage, dendritic cell, or a neutrophil, comprising administering an effective amount of composition of any one of the present embodiments to a patient in need thereof or contacting cells with a composition of any one of the present embodiments (e.g. ex vivo, e.g. with allogeneic or autologous cells). Reduction of engulfment of apoptotic cells can promote and increase anti-tumor immunity and induce immunogenic cell death. Reduction or inhibition of engulfment or phagocytosis refers to a statistically significant reduction of uptake, engulfment, or phagocytosis. Such decreases or reductions can include, for example, at least about a 5%, about 10%, about 20%, about 30%, about 40%, about 50%, about 60%, about 70%, about 80%, about 90%, about 95%, about 100% or more decrease in the measured or observed level of uptake, engulfment or phagocytosis.

In addition, this document also provides pharmaceutical compositions that comprise compositions as described herein, in combination with a pharmaceutically acceptable carrier. A “pharmaceutically acceptable carrier” (also referred to as an “excipient” or a “carrier”) is a pharmaceutically acceptable solvent, suspending agent, stabilizing agent, or any other pharmacologically inert vehicle for delivering one or more therapeutic compounds to a subject (e.g., a mammal, such as a human, non-human primate, dog, cat, sheep, pig, horse, cow, mouse, rat, or rabbit), which is nontoxic to the cell or subject being exposed thereto at the dosages and concentrations employed. Pharmaceutically acceptable carriers can be liquid or solid, and can be selected with the planned manner of administration in mind so as to provide for the desired bulk, consistency, and other pertinent transport and chemical properties, when combined with one or more of therapeutic compounds and any other components of a given pharmaceutical composition. Typical pharmaceutically acceptable carriers that do not deleteriously react with amino acids include, by way of example and not limitation: water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate). Pharmaceutically acceptable carriers also include aqueous pH buffered solutions or liposomes (small vesicles composed of various types of lipids, phospholipids and/or surfactants which are useful for delivery of a drug to a mammal). Further examples of pharmaceutically acceptable carriers include buffers such as phosphate, citrate, and other organic acids, antioxidants such as ascorbic acid, low molecular weight (less than about 10) residues) polypeptides, proteins such as serum albumin, gelatin, or immunoglobulins, hydrophilic polymers such as polyvinylpyrrolidone, amino acids such as glycine, glutamine, asparagine, arginine or lysine, monosaccharides, disaccharides, and other carbohydrates including glucose, mannose or dextrins, chelating agents such as EDTA, sugar alcohols such as mannitol or sorbitol, salt-forming counterions such as sodium, and/or nonionic surfactants such as TWEEN™, polyethylene glycol (PEG), and PLURONICS™.

Pharmaceutical compositions can be formulated by mixing one or more active agents with one or more physiologically acceptable carriers, diluents, and/or adjuvants, and optionally other agents that are usually incorporated into formulations to provide improved transfer. delivery, tolerance, and the like. A pharmaceutical composition can be formulated, e.g., in lyophilized formulations, aqueous solutions, dispersions, or solid preparations, such as tablets, dragees or capsules. A multitude of appropriate formulations can be found in the formulary known to all pharmaceutical chemists: Remington's Pharmaceutical Sciences (18th ed, Mack Publishing Company. Easton, PA (1990)), particularly Chapter 87 by Block, Lawrence, therein. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies as described herein, provided that the active agent in the formulation is not inactivated by the formulation and the formulation is physiologically compatible and tolerable with the route of administration. See, also, Baldrick, Regul Toxicol Pharmacol 32:210-218, 2000; Wang, Int J Pharm 203:1-60, 2000; Charman J Pharm Sci 89:967-978, 2000; and Powell et al. PDA J Pharm Sci Technol 52:238-311, 1998), and the citations therein for additional information related to formulations, excipients and carriers well known to pharmaceutical chemists.

Pharmaceutical compositions include, without limitation, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Compositions and formulations can contain sterile aqueous solutions, which also can contain buffers, diluents and other suitable additives (e.g., penetration enhancers, carrier compounds and other pharmaceutically acceptable carriers). Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions provided herein, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the polypeptide components within the compositions provided herein. The formulations can be sterilized if desired.

In some embodiments, a composition containing a composition as provided herein can be in the form of a solution or powder with or without a diluent to make an injectable suspension. The composition may contain additional ingredients including, without limitation, pharmaceutically acceptable vehicles, such as saline, water, lactic acid, mannitol, or combinations thereof, for example.

Any appropriate method can be used to administer a composition as described herein to a mammal. Administration can be, for example, parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). In some embodiments, administration can be topical (e.g., transdermal, sublingual, ophthalmic, or intranasal), pulmonary (e.g., by inhalation or insufflation of powders or aerosols), or oral. In addition, a composition containing a composition as described herein can be administered prior to, after, or in lieu of surgical resection of a tumor.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising.” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

This disclosure is further illustrated by the following non-limiting examples.

### EXAMPLES

### Example 1: Peptide Inhibition of Phagocytosis and Autophagy Reduction

In the experiments of this example, peptides based on the CDR3 regions of antibodies were developed that specifically bind to the T-cell immunoglobulin and mucin domain containing (Tim-4). These experiments demonstrate, in part, how the synthesized peptides inhibit phagocytosis and reduce autophagosome formation.

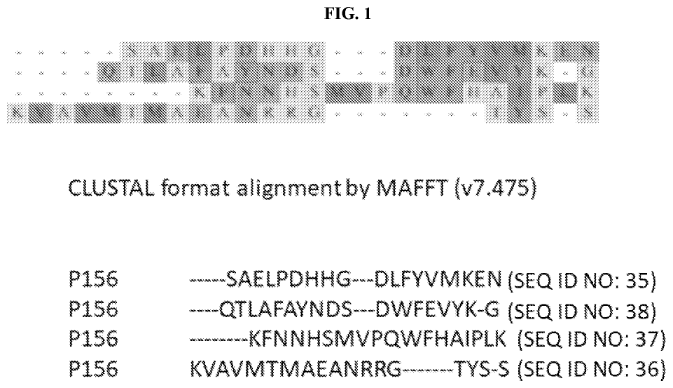

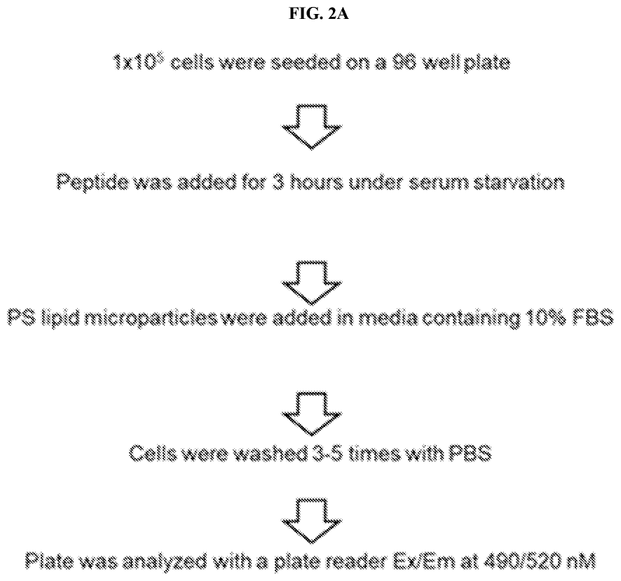

FIG. 1 shows a CLUSTAL alignment of identified CDR3 regions of antibodies that bound to Tim-4. The CDR3 regions of antibodies were then synthesized into peptides (SEQ ID NO: 7, SEQ ID NO: 11, SEQ ID NO: 3, and SEQ ID NO: 15), and the synthesized peptides were subsequently used in a phagocytosis assay and an autophagy assay to assess function. The synthesized peptides can be incorporated into the CDR3 binding region of an antibody (e.g., a single-chain antibody) to specifically bind to Tim-4.

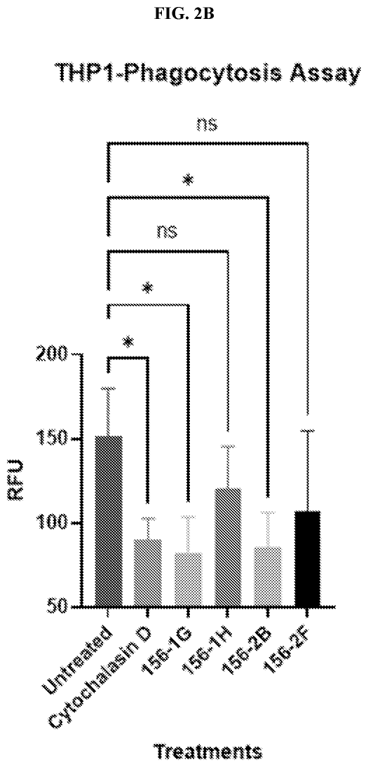

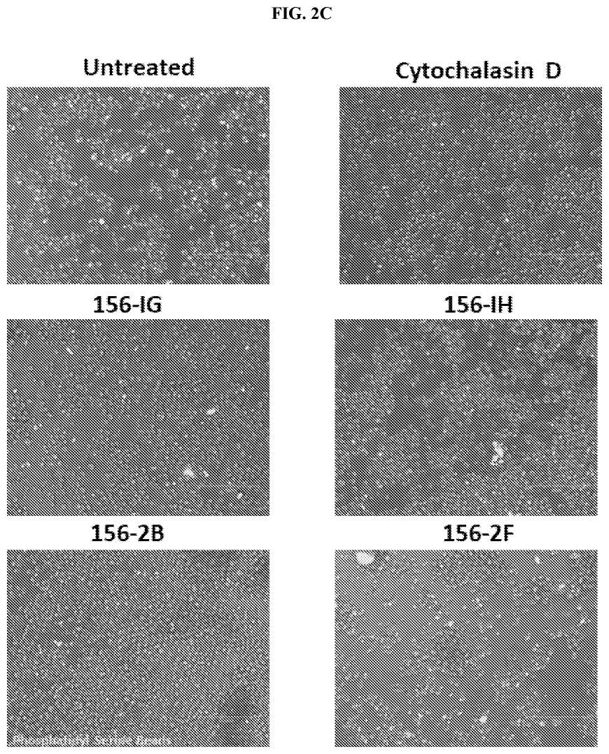

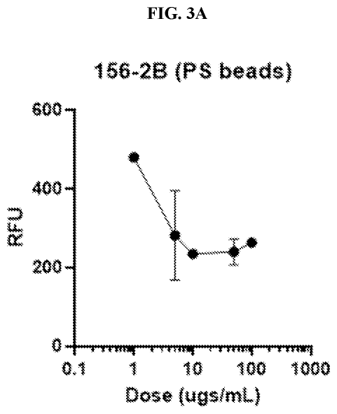

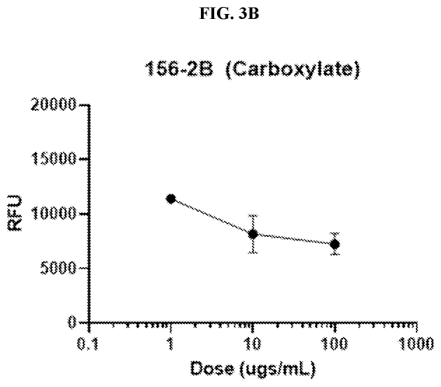

Phagocytosis assays using the developed peptides derived from the CDR3 regions of antibodies (e.g., SEQ ID NO: 35, SEQ ID NO: 36, SEQ ID NO: 37, and SEQ ID NO: 38) and THP-1 cells were completed by following the experimental outline shown in FIG. 2A. In these experiments, 1×10⁵ cells were seeded on a 96 well plate, and the synthesized peptides (e.g., SEQ ID NO: 35, SEQ ID NO: 36, SEQ ID NO: 37, and SEQ ID NO: 38) were then added onto the plate for three hours under serum starvation. Phosphatidylserine lipid microparticles were then added in media containing 10% FBS. Cells were then washed 3 to 5 times with PBS, and the level of fluorescence (e.g., relative fluorescence units, “RFU”) was then analyzed with a plate reader. In these experiments, cytochalasin D (20 μM) was used as a control to show phagocytosis failure. As shown in FIG. 2B, SEQ ID NO: 3 significantly inhibited phagocytosis in THP-1 cells. The inhibition of phagocytosis in THP-1 cells by the peptides derived from the CDR3 regions of antibodies (e.g., SEQ ID NO: 35, SEQ ID NO: 36, SEQ ID NO: 37, and SEQ ID NO: 38) is also shown visually in microscopic images (FIG. 2C). Dose response curves of SEQ ID NO: 17 and phagocytosis assays, which were completed in THP-1 cells, is shown for phosphatidylserine lipid microparticles (FIG. 3A), and carboxylate beads (FIG. 3B). In these experiments, phosphatidylserine lipid microparticles, which have the natural ligand for Tim4, showed greater inhibition phagocytosis in THP-1 cells compared to carboxylate beads. These experiments demonstrate the synthesized peptides, derived from the CDR3 regions of antibodies that specifically bind to Tim-4, inhibit phagocytosis in THP-1 cells.

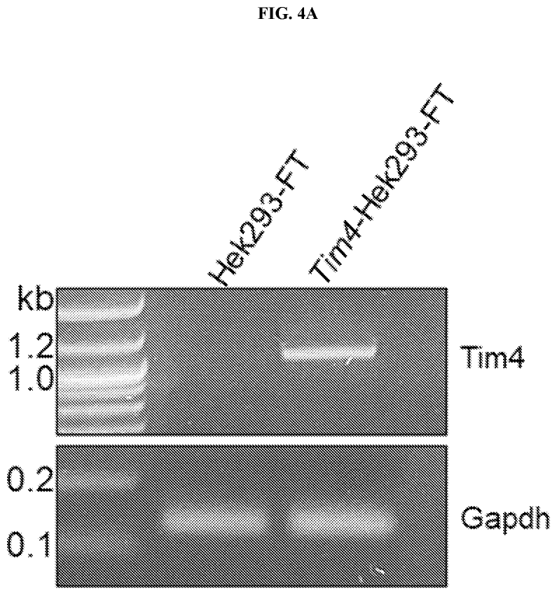

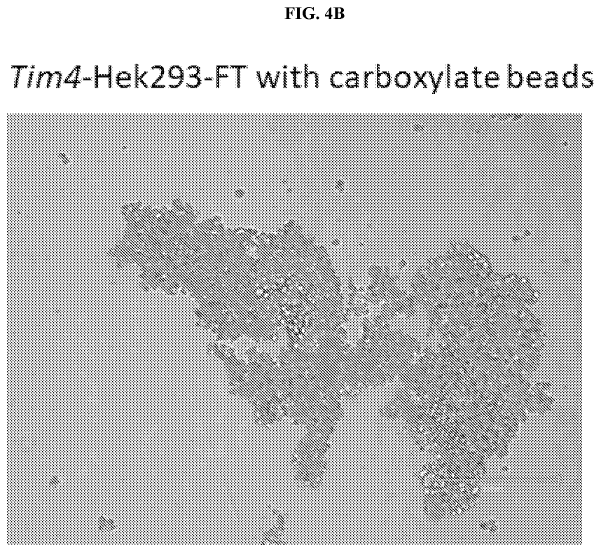

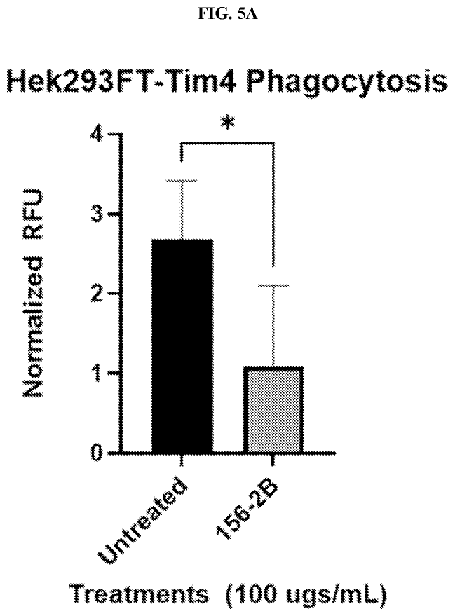

To further validate the function of the synthesized peptides, a Tim4-Hek293-FT cell line was developed (FIG. 4A). FIG. 4A shows HEK293-FT cells infected with lentiviral Tim-4 particles, and FIG. 4B is a microscopic image of Tim4-Hek293-FT cells undergoing phagocytosis using carboxylate beads. Phagocytosis assays in the Tim4-Hek293-FT cell line were performed as shown in FIG. 2A, and these experiments confirmed that SEQ ID NO: 3, derived from the CDR3 regions of antibodies that specifically bind to Tim-4, inhibit phagocytosis in the Tim4-Hek293-FT cell line (FIG. 5A, FIG. 5B). Together, these experiments demonstrate that the synthesized peptides inhibit phagocytosis, as demonstrated in the preceding experiments in both THP-1 cells and Tim4-Hek293-FT cells.

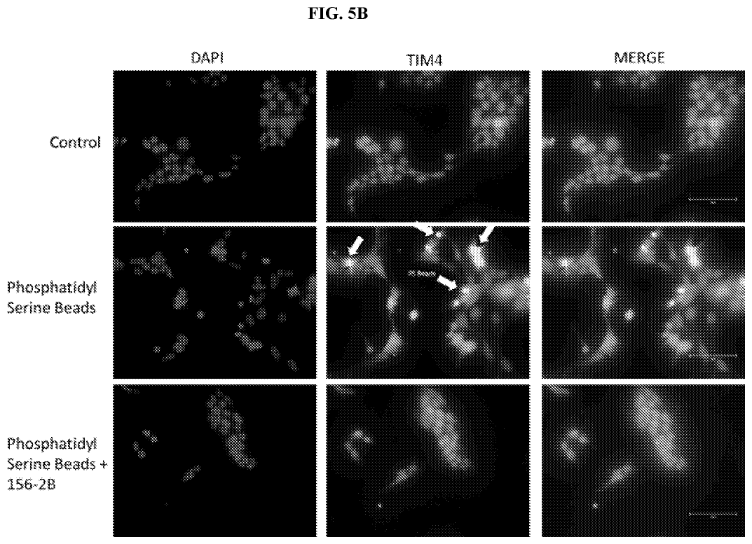

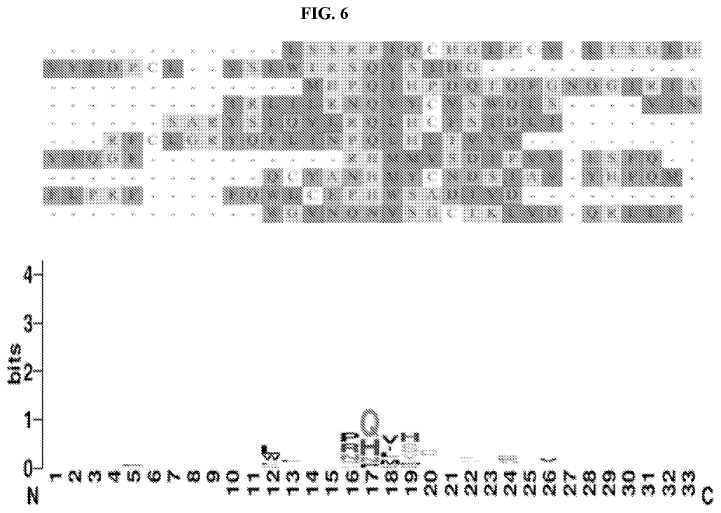

Additional peptides were also synthesized that specifically bind to Tim-4. FIG. 6 shows a CLUSTAL alignment of 20mer peptides, and these peptides (e.g., SEQ ID NO: 17) were subsequently used in a Phagocytosis assay and an Autophagy assay to assess function. The synthesized peptides can be incorporated into the CDR3 binding region of an antibody (e.g., a single-chain antibody) to specifically bind to Tim-4.

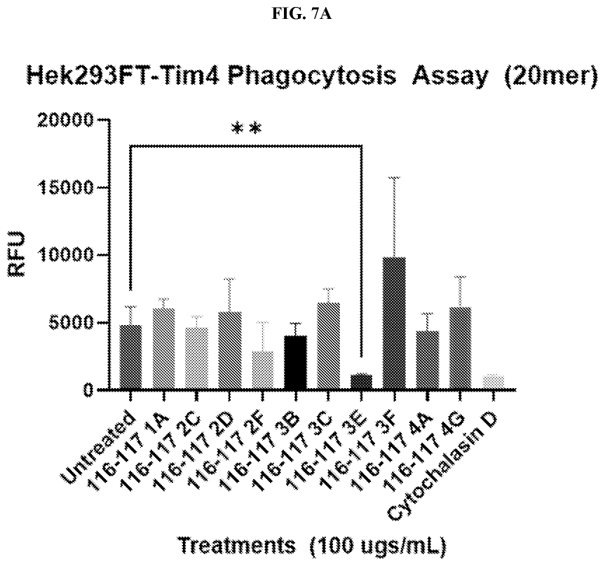

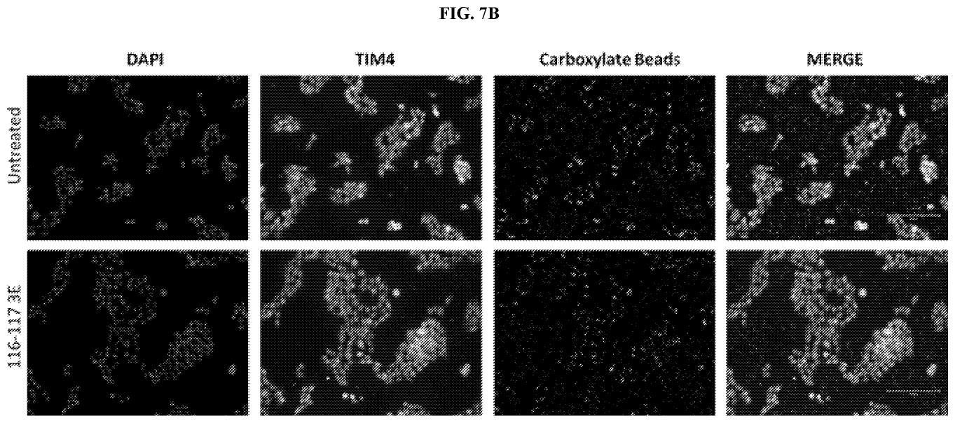

A phagocytosis assay, as previously described above and following the experimental outline of FIG. 2A, was performed in the Tim4-Hek293-FT cells. In these experiments, cytochalasin D (20 μM) was used as a control to show phagocytosis failure. As shown in FIG. 7A, SEQ ID NO: 17 inhibited phagocytosis in the Tim4-Hek293-FT cells. The inhibition of phagocytosis by SEQ ID NO: 17 is also shown in the immunofluorescence images of FIG. 7B. These experiments demonstrate that SEQ ID NO: 17, derived from the CDR3 regions of antibodies that specifically bind to Tim-4, inhibit phagocytosis in Tim4-Hek293-FT cells.

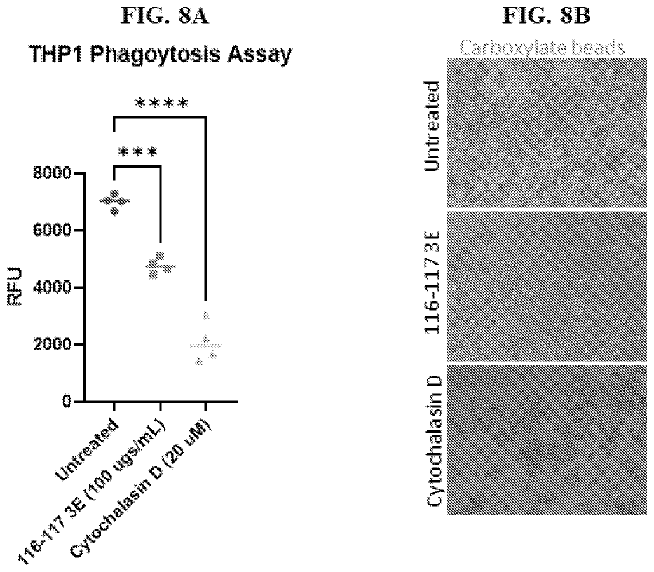

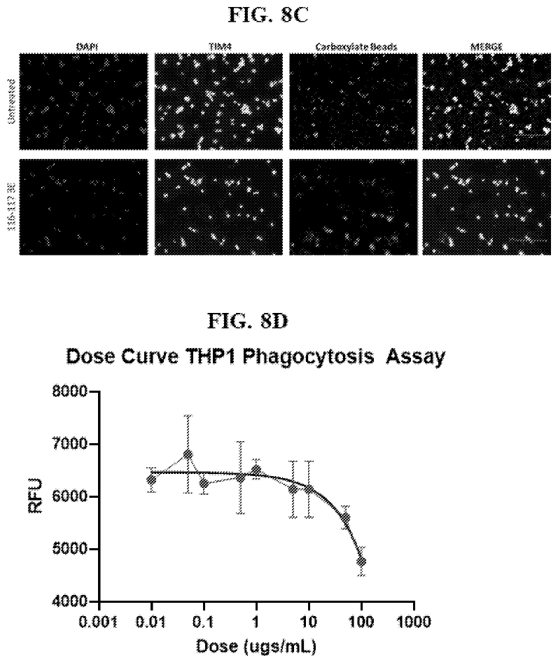

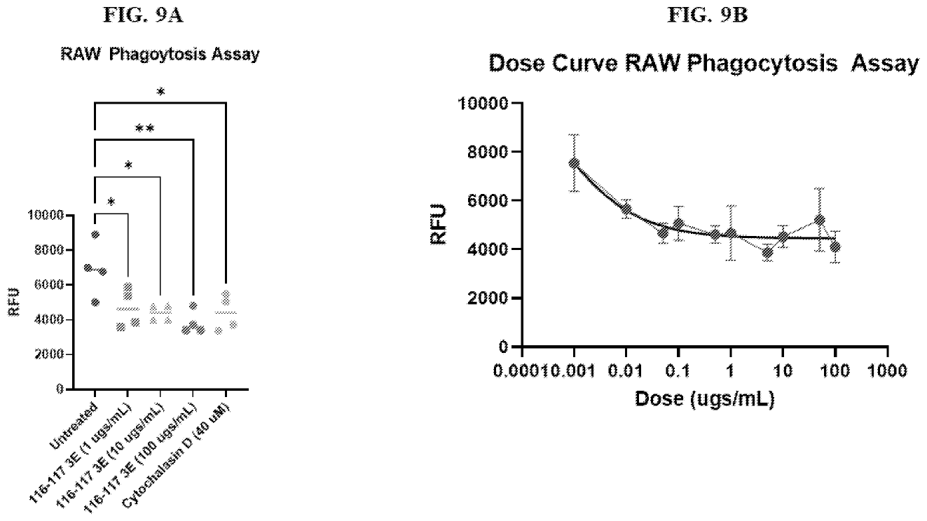

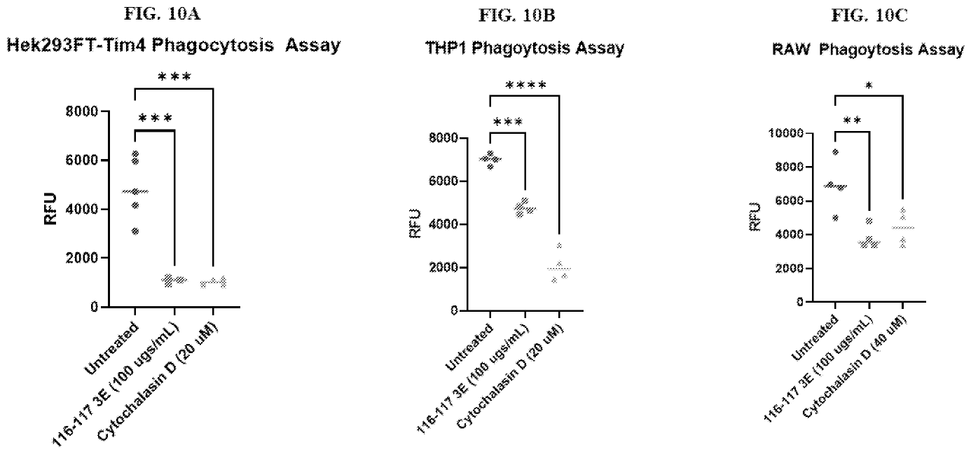

To further validate the function of the synthesized peptide, SEQ ID NO: 17, a phagocytosis assay using SEQ ID NO: 17 in THP-1 cells (FIG. 8A, FIG. 8B, FIG. 8C, and FIG. 8D), and in RAW 264.7 cells (FIG. 9A and FIG. 9B), was performed. As shown in FIG. 8A, SEQ ID NO: 17 inhibited phagocytosis in THP-1 cells, which is shown visually in FIG. 8B and FIG. 8C. A dose response curve for SEQ ID NO: 17 in THP-1 cells was generated (FIG. 8D). Further, as shown in FIG. 9A, SEQ ID NO: 17 inhibited phagocytosis in RAW 264.7 cells across a range of does. A dose response curve for SEQ ID NO: 17 in RAW 264.7 cells was generated (FIG. 9B). As shown in each of these experiments, SEQ ID NO: 17 inhibited phagocytosis in three independent cell lines: (1) Tim4-Hek293-FT cells (FIG. 10A), THP-1 cells (FIG. 10B), and RAW 264.7 cells (FIG. 10C).

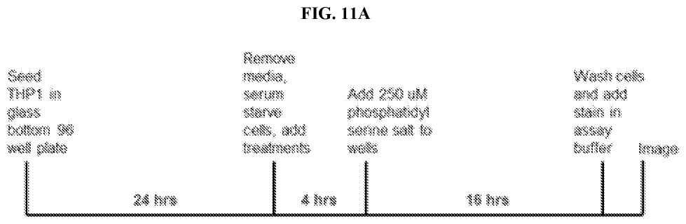

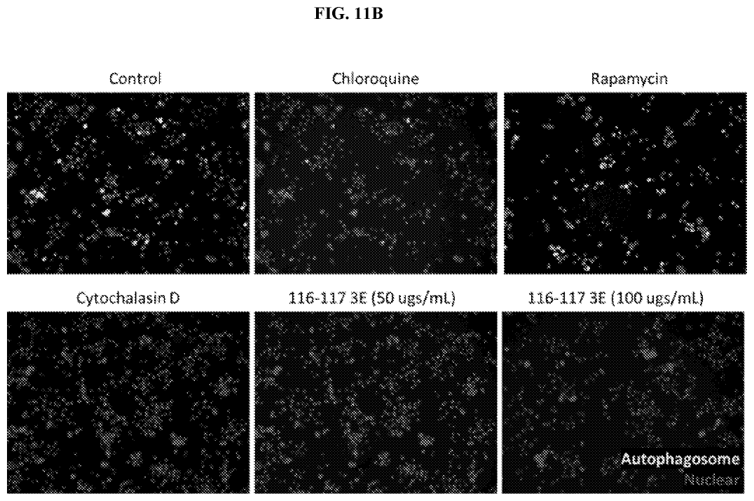

FIG. 11A and FIG. 11B show how SEQ ID NO: 17 reduces autophagosome formation in THP-1 cells. FIG. 11A shows the experimental workflow, and FIG. 11B shows representative images.

Together, these experiments demonstrate that, inter alia, peptides derived from the present CDR3 regions of the present antibodies that specifically bind to Tim-4, inhibit phagocytosis and reduce autophagosome formation.

Collectively, the experiments described in the examples above demonstrate the unexpected results of a composition that specifically binds to Tim-4, and how the synthesized peptides inhibit phagocytosis and reduce autophagosome formation.

All of the features disclosed herein may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A composition which specifically binds to T-cell immunoglobulin and mucin domain containing 4 (Tim-4), wherein the composition comprises:
(a) a single-chain antibody, or fragment thereof comprising CDR1, CDR2, and CDR3, wherein:

| | CDR1 is |
|---|---|
| (SEQ ID NO: 1) | |
| | FWTEITDVNR |
| | or |
| (SEQ ID NO: 5) | |
| | FWTEITDVNR, |
| | or |
| (SEQ ID NO: 9) | |
| | WMPVHDFSNF, |
| | or |
| (SEQ ID NO: 13) | |
| | FWTEITDVNR, or a variant thereof, |
| | CDR2 is |
| (SEQ ID NO: 2) | |
| | YETTTPSWNL |
| | or |
| (SEQ ID NO: 6) | |
| | WPGLFYAYDS, |
| | or |
| (SEQ ID NO: 10) | |
| | EKFNMNPSDN, |
| | or |
| (SEQ ID NO: 14) | |
| | YETTTPSWNL, or a variant thereof, |
| | and |
| | CDR3 is |
| (SEQ ID NO: 3) | |
| | KVAVMTMAEANRRGTYSS |
| | or |
| (SEQ ID NO: 7) | |
| | QTLAFAYNDSDWFEVYKG, |
| | or |
| (SEQ ID NO: 11) | |
| | KFNNHSMVPQWFHAIPLK, |
| | or |
| (SEQ ID NO: 15) | |
| | SAELPDHHGDLFYVMKEN, |

or a variant thereof; or
(b) a peptide, the peptide selected from:
(SEQ ID NO: 17)
QCYANHMYCNDSIAVYHFQM, or a variant thereof;
(SEQ ID NO: 22)
LSSRPIQCHGLPCVLTSGLG, or a variant thereof;
(SEQ ID NO: 30)
MHPQIHPDQTQFGNQGIRIA, or a variant thereof,
(SEQ ID NO: 23)
IRLILRNQVYCVSWQLSVIN, or a variant thereof;
(SEQ ID NO: 24)
FLPRFFQWLCEPHWSADIVD, or a variant thereof;
(SEQ ID NO: 25)
VYLDPCLVSLWTRSQVSIDG, or a variant thereof;
(SEQ ID NO: 26)
WGVNQNVSGCTKLVDQRLLF, or a variant thereof;
(SEQ ID NO: 27)
YIQGFRHMMVSDIPVVESFQ, or a variant thereof;
(SEQ ID NO: 28)
SARYSLQVLRQLHCFSIDLI, or a variant thereof;
(SEQ ID NO: 29)
RFCLGRYQFLINPQLHLTVYV, or a variant thereof;
(SEQ ID NO: 35)
SAELPDHHGDLFYVMKEN, or a variant thereof;
(SEQ ID NO: 36)
KVAVMTMAEANRRGTYSS, or a variant thereof;
(SEQ ID NO: 37)
KFNNHSMVPQWFHAIPLK, or a variant thereof;
(SEQ ID NO: 38)
QTLAFAYNDSDWFEVYKG, or a variant thereof.

2. The composition of claim 1, wherein:
CDR1 is FWTEITDVNR (SEQ ID NO: 1), or a variant thereof,
CDR2 is YETTTPSWNL (SEQ ID NO: 2), or a variant thereof, and
CDR3 is KVAVMTMAEANRRGTYSS (SEQ ID NO: 3), or a variant thereof,
wherein the variant comprises about 1, or about 2, or about 3, or about 4, or about 5 mutations, the mutations selected from substitutions or deletions.

3. The composition of claim 1, wherein
CDR1 is FWTEITDVNR (SEQ ID NO: 5), or a variant thereof,
CDR2 is WPGLFYAYDS (SEQ ID NO: 6), or a variant thereof, and
CDR3 is QTLAFAYNDSDWFEVYKG (SEQ ID NO: 7), or a variant thereof,
wherein the variant comprises about 1, or about 2, or about 3, or about 4, or about 5 mutations, the mutations selected from substitutions or deletions.

4. The composition of claim 1, wherein:
CDR1 is WMPVHDFSNF (SEQ ID NO: 9), or a variant thereof,
CDR2 is EKFNMNPSDN (SEQ ID NO: 10), or a variant thereof, and
CDR3 is KFNNHSMVPQWFHAIPLK (SEQ ID NO: 11), or a variant thereof,
wherein the variant comprises about 1, or about 2, or about 3, or about 4, or about 5 mutations, the mutations selected from substitutions or deletions.

5. The composition of claim 1, wherein:
CDR1 is FWTEITDVNR (SEQ ID NO: 13), or a variant thereof,
CDR2 is YETTTPSWNL (SEQ ID NO: 14), or a variant thereof, and
CDR3 is SAELPDHHGDLFYVMKEN (SEQ ID NO: 15), or a variant thereof,
wherein the variant comprises about 1, or about 2, or about 3, or about 4, or about 5 mutations, the mutations selected from substitutions or deletions.

6. The composition of claim 1, wherein the single-chain antibody, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

7. The composition of claim 6, wherein the variable region FW sequences are human.

8. The composition of claim 1 or 2, wherein the single-chain antibody comprises an amino acid sequence of:
QVQLVESGGGVVQPGRSLRLSCAASFWTEITDVNRWFRQAPGKEREFVAYETTT PSWNLYADSVKGRFTISRDNSKNTLYLQMNSLRAEDTAVYYCARKVAV MTMAEANRRGTYSSWGQGTLVTVSSGPGGQ (SEQ ID NO: 4), or
an amino acid sequence having at least about 90%, or at least about 95%, or at least about 97%, or at least about 98%, or at least about 99% identity with SEQ ID NO: 4.

9. The composition of claim 1 or 3, wherein the single-chain antibody comprises an amino acid sequence of:
QVQLVESGGGVVQPGRSLRLSCAASWMPVHDFSNFWFRQAPGKEREFVAEKFN MNPSDNYADSVKGRFTISRDNSKNTLYLQMNSLRAEDTAVYYCARKFNN HSMVPQWFHAIPLKWGQGTLVTVSSGPGGQ (SEQ ID NO: 8), or
an amino acid sequence having at least about 90%, or at least about 95%, or at least about 97%, or at least about 98%, or at least about 99% identity with SEQ ID NO: 8.

10. The composition of claim 1 or 4, wherein the single-chain antibody comprises an amino acid sequence of:
QVQLVESGGGVVQPGRSLRLSCAASFWTEITDVNRWFRQAPGKEREFVAWPGL FYAYDSYADSVKGRFTISRDNSKNTLYLQMNSLRAEDTAVYYCARQTLA FAYNDSDWFEVYKGWGQGTLVTVSSGPGGQ (SEQ ID NO: 12), or
an amino acid sequence having at least about 90%, or at least about 95%, or at least about 97%, or at least about 98%, or at least about 99% identity with SEQ ID NO: 12.

11. The composition of claim 1 or 5, wherein the single-chain antibody comprises an amino acid sequence of:
QVQLVESGGGVVQPGRSLRLSCAASFWTEITDVNRWFRQAPGKEREFVAYETTT PSWNLYADSVKGRFTISRDNSKNTLYLQMNSLRAEDTAVYYCARSAELP DHHGDLFYVMKENWGQGTLVTVSSGPGGQ (SEQ ID NO: 16), or
an amino acid sequence having at least about 90%, or at least about 95%, or at least about 97%, or at least about 98%, or at least about 99% identity with SEQ ID NO: 16.

12. The composition of claim 1, wherein the peptide is:
QCYANHMYCNDSIAVYHFQM (SEQ ID NO: 17), or a variant thereof,
wherein the variant comprises about 1, or about 2, or about 3, or about 4, or about 5 mutations, the mutations selected from substitutions or deletions,
SAELPDHHGDLFYVMKEN (SEQ ID NO: 35), or a variant thereof; wherein the variant comprises about 1, or about 2, or about 3, or about 4, or about 5 mutations, the mutations selected from substitutions or deletions, or
KVAVMTMAEANRRGTYSS (SEQ ID NO: 36), or a variant thereof; wherein the variant comprises about 1, or about 2, or about 3, or about 4, or about 5 mutations, the mutations selected from substitutions or deletions, or
KFNNHSMVPQWFHAIPLK (SEQ ID NO: 37), or a variant thereof; wherein the variant comprises about 1, or about 2, or about 3, or about 4, or about 5 mutations, the mutations selected from substitutions or deletions, or
QTLAFAYNDSDWFEVYKG (SEQ ID NO: 38), or a variant thereof; wherein the variant comprises about 1, or about 2, or about 3, or about 4, or about 5 mutations, the mutations selected from substitutions or deletions.

13. The composition of claim 1, wherein the peptide is:
LSSRPIQCHGLPCVLTSGLG (SEQ ID NO: 22), or a variant thereof;
wherein the variant comprises about 1, or about 2, or about 3, or about 4, or about 5 mutations, the mutations selected from substitutions or deletions, or
MHPQIHPDQTQFGNQGIRIA (SEQ ID NO: 30), or a variant thereof;
wherein the variant comprises about 1, or about 2, or about 3, or about 4, or about 5 mutations, the mutations selected from substitutions or deletions, or
IRLILRNQVYCVSWQLSVIN (SEQ ID NO: 23), or a variant thereof;
wherein the variant comprises about 1, or about 2, or about 3, or about 4, or about 5 mutations, the mutations selected from substitutions or deletions.

14. The composition of claim 1, wherein the peptide is:
FLPRFFQWLCEPHWSADIVD (SEQ ID NO: 24), or a variant thereof,
wherein the variant comprises about 1, or about 2, or about 3, or about 4, or about 5 mutations, the mutations selected from substitutions or deletions, or
VYLDPCLVSLWTRSQVSIDG (SEQ ID NO: 25), or a variant thereof,
wherein the variant comprises about 1, or about 2, or about 3, or about 4, or about 5 mutations, the mutations selected from substitutions or deletions, or
WGVNQNVSGCTKLVDQRLLF (SEQ ID NO: 26), or a variant thereof,
wherein the variant comprises about 1, or about 2, or about 3, or about 4, or about 5 mutations, the mutations selected from substitutions or deletions.

15. The composition of claim 1, wherein the peptide is:
YIQGFRHMMVSDIPVVESFQ (SEQ ID NO: 27), or a variant thereof,
wherein the variant comprises about 1, or about 2, or about 3, or about 4, or about 5 mutations, the mutations selected from substitutions or deletions,
SARYSLQVLRQLHCFSIDLI (SEQ ID NO: 28), or a variant thereof,
wherein the variant comprises about 1, or about 2, or about 3, or about 4, or about 5 mutations, the mutations selected from substitutions or deletions,
RFCLGRYQFLINPQLHLTVYV (SEQ ID NO: 29), or a variant thereof,
wherein the variant comprises about 1, or about 2, or about 3, or about 4, or about 5 mutations, the mutations selected from substitutions or deletions.

16. The composition of any one of claim 1 or 12-15, wherein the peptide further comprises an additional peptide.

17. The composition of claim 16, wherein the composition comprises a dimer of peptides.

18. The composition of claim 16, wherein the composition comprises a trimer of peptides.

19. The composition of any one of claims 16-18, wherein the peptides are joined with a linker which is substantially comprised of glycine and serine residues.

20. The composition of claim 19, wherein the linker is (GGS)ₙ, wherein ₙ is 1, or 2, or 3, or 4, or 5.

21. The composition of claim 20, wherein the linker is GGSGGSGGSG (SEQ ID NO: 21), or a variant thereof,
wherein the variant comprises about 1, or about 2, or about 3, or about 4, or about 5 mutations, the mutations selected from substitutions or deletions.

22. The composition of any of the above claims, further comprising a targeting moiety.

23. The composition of claim 22, wherein the targeting moiety is directed to a tumor cell.

24. The composition of claim 23, wherein the targeting moiety is directed to a tumor-associated antigen (TAA).

25. The composition of claim 24, wherein the TAA is selected from HER2, PSA, TRP-2, EpCAM, GPC3, mesothelin (MSLN), and EGFR.

26. A polynucleotide comprising a nucleic acid sequence encoding the single-chain antibody, or a fragment thereof, or peptide of any of the above claims.

27. A vector comprising the polynucleotide of claim 26.

28. A host cell comprising the vector of claim 27.

29. A pharmaceutical composition comprising the composition of any of claims 1-28, and a pharmaceutically acceptable excipient or carrier.

30. A method for treating or preventing cancer, comprising administering an effective amount of the composition of any one of claims 1-28 to a patient in need thereof.

31. The method of claim 30, wherein the cancer is a solid tumor.

32. The method of claim 30, wherein the cancer is a blood cancer.

33. The method of any one of claims 30-32, wherein the cancer is selected form one or more of a cancer of a blood vessel, an eye tumor, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

34. A method for treating or preventing an autoimmune disease or disorder, comprising administering an effective amount of the composition of any one of claims 1-28 to a patient in need thereof.

35. The method of claim 34, wherein the autoimmune disease or disorder is selected from graft versus host disease, transplantation rejection (e.g., prevention of allograft rejection), multiple sclerosis, diabetes mellitus, lupus, celiac disease, Crohn's disease, ulcerative colitis, Guillain-Barre syndrome, scleroderma, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Meniere's syndrome; pernicious anemia, rheumatoid arthritis, systemic lupus erythematosus, dermatomyositis, Sjogren's syndrome, lupus erythematosus, multiple sclerosis, myasthenia gravis, Reiter's syndrome, and Grave's disease.

36. The method of claim 34, wherein the autoimmune disease or disorder is graft versus host disease.
